(19) United States
(12) Patent Application Publication
(10) Pub. No.: US 2019/0231884 A1
(43) Pub. Date: Aug. 1, 2019

(54) SYNTHETIC PULMONARY SURFACTANT COMPOSITION FOR TREATING LUNG CONDITIONS

(71) Applicant: STELLENBOSCH UNIVERSITY, Stellenbosch (ZA)

(72) Inventors: Lyne VAN RENSBURG, Tygerberg (ZA); Johann Martin VAN ZYL, Oakdale, Bellville (ZA)

(21) Appl. No.: 16/339,626

(22) PCT Filed: Oct. 4, 2017
(86) PCT No.: PCT/IB2017/056119

§ 371 Date: Apr. 4, 2019

(30) Foreign Application Priority Data
Oct. 4, 2016 (ZA) 2016/06808

(51) Int. Cl. A61K 47/42; A61P 11/00; A61P 35/00; A61P 37/02; A61P 31/06; A61K 47/24; A61K 47/10; A61K 47/32

(52) U.S. Cl. CPC A61K 47/42; A61P 11/00; A61P 35/00; A61K 47/32; A61P 31/06; A61K 47/24; A61K 47/10; A61P 37/02

(57) ABSTRACT

A synthetic pulmonary surfactant composition for use in the treatment of inflammatory or cell proliferation disorders of the lungs is provided. The composition comprises a lipidaceous carrier and a peptide complex of poly-L-lysine or a pharmaceutically acceptable salt thereof and poly-L-glutamic acid or poly-L-aspartic acid or a pharmaceutically acceptable salt thereof, the peptide complex having a charge-neutralised region and a positively-charged region. The synthetic pulmonary surfactant composition may be provided in an inhalable formulation. The synthetic pulmonary surfactant composition may also be combined with a drug for use in treating a lung infection, particularly a lung infection characterised by inflammation in the lungs so that the combination provides dual immunomodulatory effects.

### CROSS-REFERENCE(S) TO RELATED APPLICATIONS

This application claims priority from South African provisional patent application number 2016/06808 filed on 4 Oct. 2016, which is incorporated by reference herein.

### FIELD OF THE INVENTION

This invention relates to an exogenous synthetic pulmonary surfactant composition. In particular, the invention relates to the use of the synthetic pulmonary surfactant composition as an anti-inflammatory and/or chemotherapeutic agent. The synthetic pulmonary surfactant composition having an anti-inflammatory effect can also be used as a drug delivery agent with dual immunomodulatory effects.

### BACKGROUND TO THE INVENTION

Pulmonary surfactants are found at the alveolar surface and are essential for breathing. They consist of a complex mixture of phospholipids (85%), neutral lipids (5%), and several specific surfactant proteins (5%) which reduce surface tension at the alveolar surface, allowing for rapid gaseous exchange. The unique spreading properties of the pulmonary surfactant reduce surface tension, thereby promoting lung expansion (also known as compliance) during inspiration, and preventing lung collapse during expiration.

Without surfactant, the air sacs or alveoli of the lungs collapse and are unable to absorb sufficient oxygen. This can manifest as an inhibition of gas exchange in the lungs, causing a condition known as hyaline membrane disease (HMD), also known as respiratory distress syndrome (RDS). This condition occurs most frequently in premature infants, but also often occurs in older children and adults. The observation that preterm infants with RDS suffer from an alveolar surface-active material deficiency led to the treatment of the condition with exogenous surfactant replacements.

Various exogenous pulmonary surfactants are commercially available, such as those listed below in Table 1. These include mammalian-derived or natural surfactants containing surfactant proteins and synthetic protein-free lipid mixtures.

TABLE 1
A selection of commercially available pulmonary surfactants

| Generic name | Brand name | Manufacturer |
|---|---|---|
| Beractant | Survanta ® | Abbott Laboratories (USA) |
| Surfactant-TA | Surfacten ® | Tokoyo Tanabe (Japan) |
| Porcine surfactant | Curosurf ® | Chiesi Pharmaceuticals (Italy) |
| Calf pulmonary s | Infasurf ® | Forest Laboratories (USA) |
| urfactant (CLSE) | | |
| SF-RI 1 | Alveofact ® | Boehringer (Germany) |
| Artificial lung | Pneumactant ® | Britannia Pharmaceuticals (UK) |
| expanding compound | | |
| (ALEC) | | |
| Colfosceril palmitate | Exosurf ® | Glaxo Wellcome Co (USA) |
| hexadecanol, tyloxapol | | |
| Bovine lipid | Liposurf ® | Cipla Limited (India) |
| extract surfactant | | |

Mammalian-derived surfactant, also referred to herein as native or natural pulmonary surfactant, consists mainly of phospholipids, the major phospholipid being dipalmitoyl phosphatidylcholine (DPPC). It also includes phosphatidyl glycerol (PG) and surfactant proteins (SP) A, B, C, and D. Mammalian-derived surfactants have been available for many years, but are expensive and their therapeutic application has been focused upon use in HMD/RDS occurrence in premature infants. These surfactant formulations usually contain proteins derived from bovine or porcine sources and hence pose a potential risk for the transmission of animal-associated pathogens and allergic responses.

Reconstituted surfactants usually consist of a lipidaceous carrier and added hydrophobic proteins, either isolated from animal tissues or obtained through recombinant techniques. Synthetic surfactants include synthetic peptidic derivatives of proteins that may act to mimic natural surfactant proteins.

Generally the properties, potential therapeutic activity and use of exogenous mammalian-derived surfactants, reconstituted surfactants and synthetic surfactants depend on their composition, including the particular phospholipid mixture and the peptide or protein components. Traditionally exogenous surfactants have been administered directly into the lungs of new-borns, children and adults via intubation or instillation. New less invasive methods are being developed for the administration of exogenous surfactants which makes the use of surfactants in medical therapies and treatment more attractive.

The preceding discussion of the background to the invention is intended only to facilitate an understanding of the present invention. It should be appreciated that the discussion is not an acknowledgment or admission that any of the material referred to was part of the common general knowledge in the art as at the priority date of the application.

### SUMMARY OF THE INVENTION

In accordance with the invention, there is provided a synthetic pulmonary surfactant composition for use in the treatment of inflammatory or cell proliferation disorders of the lungs, the composition comprising a lipidaceous carrier and a peptide complex of poly-L-lysine or a pharmaceutically acceptable salt thereof and poly-L-glutamic acid or poly-L-aspartic acid or a pharmaceutically acceptable salt thereof, the peptide complex having a charge-neutralised region and a positively-charged region.

Still further features provide for the salt of poly-L-lysine to be poly-L-lysine.HBr; for the poly-L-lysine.HBr to be of the formula (I) and n to range from 100 to 135, preferably from 103 to 135, and more preferably from 103 to 119

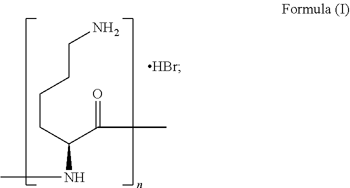

for the salt of poly-L-glutamic acid to be poly-L-glutamic acid sodium salt; for the poly-L-glutamic acid sodium salt to be of the formula (II) and x is at least 50, preferably at least 68, and more preferably at least 86

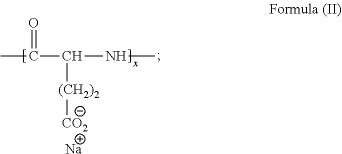

and for the poly-L-lysine chain to be longer than the poly-L-glutamic acid or poly-L-aspartic acid chain by at least 17 residues, preferably by at least 50 residues and more preferably by at least 85 residues.

Yet further features provide for the synthetic pulmonary surfactant composition to comprise
dipalmitoyl phosphatidylcholine (DPPC);
phosphatidylglycerol (PG);
hexadecanol;
tyloxapol;

poly-L-lysine.HBr;
poly-L-glutamic acid sodium salt;
sodium chloride; and
a pharmaceutically acceptable carrier.

Further features provide for the treatment of inflammatory or cell proliferation disorders of the lungs to occur by the inhibition of the secretion of pro-inflammatory cytokines by alveolar macrophages in the presence of the surfactant composition; and for the pro-inflammatory cytokines to be TNF-α, IL-1β, IL-6 and KC/GRO.

Still further features provide for the cell proliferation disorder to be lung cancer; for the cancer to be a lung adenocarcinoma; for a therapeutically effective amount of the surfactant composition to depend on the dosage of the surfactant composition and the exposure time of the lung cancer to the surfactant composition; for a therapeutically effective concentration of the surfactant composition to be 500 μg/ml or more when the exposure time is 1 hour or more; and for the synthetic pulmonary surfactant composition to include a further chemotherapeutic agent to be co-administered with the surfactant composition.

A further feature provides for the synthetic pulmonary surfactant composition to include an antimicrobial agent to be co-administered with the surfactant composition, preferably an antibiotic, more preferably an oxazolidinone, more preferably Linezolid.

The invention further provides for the use of a surfactant composition as described above in the manufacture of a medicament for the treatment of inflammatory or cell proliferation disorders of the lungs.

The invention yet further provides for a method of treating inflammatory or cell proliferation disorders of the lungs in a subject or patient which includes administering to the subject or patient in need thereof a therapeutically effective amount of a surfactant composition as described above.

Further features provide for a therapeutically effective amount of the surfactant composition to be administered to the lungs via intubation, direct pulmonary administration or inhalation, preferably by inhalation; for the surfactant composition to be in an inhalable formulation; and for a pressurised meter dose inhaler to be used to administer a therapeutically effective amount of the surfactant composition.

Furthermore, an inhalable composition for use in the treatment of inflammatory or cell proliferation disorders of the lungs is provided which includes a surfactant composition as defined above as an active component or ingredient.

Further features provide for the inhalable composition to optionally include one or more further excipients selected from a carrier, a propellant, a solubiliser, a preservative and a stabiliser; for the inhalable composition to be formulated for nebulisation, formulated to form a liquid aerosol or formulated to be an inhalable powder; for the inhalable composition to include two or more active components; for the second and further active components to be pharmaceutical compositions or compounds to be used for the treatment of a disease that results in an inflammatory or cell proliferation disorder of the lungs; for the pharmaceutical composition or compound to be a chemotherapeautic agent; and for the pharmaceutical composition or compound to be an antimicrobial, preferably an antibiotic, more preferably an oxazolidinone, even more preferably Linezolid.

In accordance with a second aspect of the invention, there is provided a synthetic pulmonary surfactant composition and a drug combination for use in treating a lung infection, the synthetic pulmonary surfactant composition comprising a lipidaceous carrier and a peptide complex of poly-L-lysine or a pharmaceutically acceptable salt thereof and poly-L-glutamic acid or poly-L-aspartic acid or a pharmaceutically acceptable salt thereof, the peptide complex having a charge-neutralised region and a positively-charged region.

Further features of this aspect provide for the salt of poly-L-lysine to be poly-L-lysine.HBr; for the poly-L-lysine.HBr to be of the formula (I) and n to range from 100 to 135, preferably from 103 to 135, and more preferably from 103 to 119

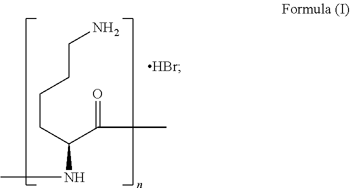

for the salt of poly-L-glutamic acid to be poly-L-glutamic acid sodium salt; for the poly-L-glutamic acid sodium salt to be of the formula (II) and x is at least 50, preferably at least 68, and more preferably at least 86

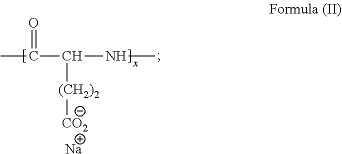

and for the poly-L-lysine chain to be longer than the poly-L-glutamic acid or poly-L-aspartic acid chain by at least 17 residues, preferably by at least 50 residues and more preferably by at least 85 residues.

Still further features of this aspect provide for the synthetic pulmonary surfactant composition to comprise
dipalmitoyl phosphatidylcholine (DPPC);
phosphatidylglycerol (PG);
hexadecanol;
tyloxapol;
poly-L-lysine.HBr;
poly-L-glutamic acid sodium salt;
sodium chloride; and
a pharmaceutically acceptable carrier.

Yet further features of this aspect provide for the lung infection to cause inflammation in the lungs; for the synthetic pulmonary surfactant composition and drug combination to provide dual immunomodulatory effects; and for the synthetic pulmonary surfactant composition to act as a permeabilising agent of cell membranes that increases permeability of the drug across the membrane.

Further features provide for the synthetic pulmonary surfactant composition and drug combination to be delivered into the lungs leading to site-specific drug delivery; for a therapeutically effective amount of the synthetic pulmonary surfactant composition and drug combination to be administered to the lungs via intubation, direct pulmonary administration or inhalation, preferably by inhalation; and for the synthetic pulmonary surfactant composition and drug combination to be in an inhalable formulation.

Still further features provide for the lung infection to be a bacterial or viral infection; for the lung infection to be associated with tuberculosis (TB), pneumonia, cystic fibrosis and/or other diseases or diseased states; for the drug to be selected from one or more of tobramycin, Isoniazid (INH), Moxifloxacin, Ofloxacin, Pyrazinamide, Linezolid, amoxicillin, and ceftazidime; for the drug to be an antibiotic effective against Gram positive bacteria; for the antibiotic to be an oxazolidinone, preferably Linezolid for use in the treatment of a Mycobacterium tuberculosis infection (TB).

The invention further provides for the use of a synthetic pulmonary surfactant composition and drug combination as described above in the manufacture of a medicament for the treatment of a lung infection.

The invention yet further provides for a method of treating a lung infection in a patient which includes administering to a patient in need thereof a therapeutically effective amount of a synthetic pulmonary surfactant composition and a drug combination as described above.

An embodiment of the invention will now be described, by way of example only, with reference to the accompanying drawings.

### BRIEF DESCRIPTION OF THE DRAWINGS

In the drawings:

FIG. 1 is a bar graph of the concentration of TNF-α in cell supernatant by NR8383 AM in the presence of (A) Curosurf®, (B) Synsurf® and (C) Liposurf® at 100-1500 μg/ml phospholipids;

FIG. 2 is a bar graph of the concentration of TNF-α in cell supernatant by lipopolysaccharide-stimulated (LPS-stimulated) NR8383 AM in the presence of (A) Curosurf®, (B) Synsurf® and (C) Liposurf® at 100-1500 μg/ml phospholipids;

FIG. 3 is a bar graph of the concentration of IL-1β in cell supernatant by LPS-stimulated NR8383 AM in the presence of (A) Curosurf®, (B) Synsurf® and (C) Liposurf® at 100-1500 μg/ml phospholipids;

FIG. 4 is a bar graph of the concentration of IL-6 in cell supernatant by LPS-stimulated NR8383 AM in the presence of (A) Curosurf®, (B) Synsurf® and (C) Liposurf® at 100-1500 μg/ml phospholipids;

FIG. 5 is a bar graph of the concentration of KC/GRO in cell supernatant by LPS-stimulated NR8383 AM in the presence of (A) Curosurf®, (B) Synsurf® and (C) Liposurf® at 100-1500 μg/ml phospholipids;

FIG. 6 is a bar graph of showing the NR8383 AM cell viability as a percentage of the untreated control cells in the presence of Curosurf®, Synsurf® and Liposurf® at comparable DPPC concentrations for a 30 min exposure time;

FIG. 7 is a bar graph of showing the NR8383 AM cell viability as a percentage of the untreated control cells in the presence of Curosurf®, Synsurf® and Liposurf® at comparable DPPC concentrations for a 1 hour exposure time;

FIG. 8 is a bar graph of showing the NR8383 AM cell viability as a percentage of the untreated control cells in the presence of Curosurf®, Synsurf® and Liposurf® at comparable DPPC concentrations for a 4 hours exposure time;

FIG. 9 is a bar graph of showing the NR8383 AM cell viability as a percentage of the untreated control cells in the presence of Curosurf®, Synsurf® and Liposurf® at comparable DPPC concentrations for a 12 hours exposure time;

FIG. 10 is a bar graph of showing the NR8383 AM cell viability as a percentage of the untreated control cells in the presence of Curosurf®, Synsurf® and Liposurf® at comparable DPPC concentrations for a 24 hours exposure time;

FIG. 11 is a bar graph of showing the A549 (lung carcinoma) cell viability as a percentage of the untreated control cells in the presence of Curosurf®, Synsurf® and Liposurf® at comparable DPPC concentrations for a 30 min exposure time;

FIG. 12 is a bar graph of showing the A549 (lung carcinoma) cell viability as a percentage of the untreated control cells in the presence of Curosurf®, Synsurf® and Liposurf® at comparable DPPC concentrations for a 1 hour exposure time;

FIG. 13 is a bar graph of showing the A549 (lung carcinoma) cell viability as a percentage of the untreated control cells in the presence of Curosurf®, Synsurf® and Liposurf® at comparable DPPC concentrations for a 4 hours exposure time;

FIG. 14 is a bar graph of showing the A549 (lung carcinoma) cell viability as a percentage of the untreated control cells in the presence of Curosurf®, Synsurf® and Liposurf® at comparable DPPC concentrations for a 12 hours exposure time;

FIG. 15 is a bar graph of showing the A549 (lung carcinoma) cell viability as a percentage of the untreated control cells in the presence of Curosurf®, Synsurf® and Liposurf® at comparable DPPC concentrations for a 24 hours exposure time;

FIG. 16 is a bar graph showing the effect of Curosurf® at different DPPC concentrations of 500 to 1500 μg/ml on un-stimulated oxidative burst measured by mean channel green fluorescence of DCF-DA with the percentage inhibition being relative to basal AM fluorescence at 100% vs Control (untreated and un-stimulated) (n=5) (one-way analysis of variance (ANOVA), Tukey's post-test ****P≤0.0001 in comparison to the control);

FIG. 17 is a bar graph showing the effect of Liposurf® at different DPPC concentrations of 500 to 1500 μg/ml on un-stimulated oxidative burst measured by mean channel green fluorescence of DCF-DA with the percentage inhibition being relative to basal AM fluorescence at 100% vs Control (untreated and un-stimulated) (n=5) (one-way analysis of variance (ANOVA), Tukey's post-test ****P≤0.0001 in comparison to the control);

FIG. 18 is a bar graph showing the effect of Synsurf® at different DPPC concentrations of 500 to 1500 μg/ml on un-stimulated oxidative burst measured by mean channel green fluorescence of DCF-DA with the percentage inhibition being relative to LPS-stimulated AM fluorescence at 100% vs Control (untreated and un-stimulated) (n=5) (one-way analysis of variance (ANOVA), Tukey's post-test ***P≤0.001 in comparison to the control);

FIG. 19 is a bar graph showing the effect of Curosurf® at different DPPC concentrations of 500 to 1500 μg/ml on LPS-stimulated oxidative burst measured by mean channel green fluorescence of DCF-DA with the percentage inhibition being relative to basal AM fluorescence at 100% (***P≤0.001 vs Control (LPS alone) (n=3) (one-way analysis of variance (ANOVA), Tukey's post-test ***P≤0.001 in comparison to the control));

FIG. 20 is a bar graph showing the effect of Liposurf® at different DPPC concentrations of 500 to 1500 μg/ml on LPS-stimulated oxidative burst measured by mean channel green fluorescence of DCF-DA with the percentage inhibition being relative to basal AM fluorescence at 100% (one-way analysis of variance (ANOVA), Tukey's post-test *P≤0.05, **P≤0.01, ***P≤0.001 in comparison to the control (LPS alone) (n=3));

FIG. 21 is a bar graph showing the effect of Synsurf® at different DPPC concentrations of 500 to 1500 μg/ml on LPS-stimulated oxidative burst measured by mean channel green fluorescence of DCF-DA with the percentage inhibition being relative to basal AM fluorescence at 100% (one-way analysis of variance (ANOVA), Tukey's post-test **P≤0.01, ***P≤0.001 in comparison to the control (LPS alone) (n=3));

FIG. 22 is a bar graph showing the effect of Curosurf® at different DPPC concentrations of 500 to 1500 μg/ml on un-stimulated oxidative burst measured by mean channel green fluorescence of DCF-DA with the percentage inhibition being relative to A549 fluorescence at 100% (one-way analysis of variance (ANOVA), Tukey's post-test *P≤0.05, **P≤0.01 in comparison to the control (LPS alone) (n=3));

FIG. 23 is a bar graph showing the effect of Liposurf® at different DPPC concentrations of 500 to 1500 μg/ml on un-stimulated oxidative burst measured by mean channel green fluorescence of DCF-DA with the percentage inhibition being relative to A549 fluorescence at 100% (one-way analysis of variance (ANOVA), Tukey's post-test **P≤0.01, ***P≤0.001 in comparison to the control (LPS alone) (n=3));

FIG. 24 is a bar graph showing the effect of Synsurf® at different DPPC concentrations of 500 to 1500 μg/ml on un-stimulated oxidative burst measured by mean channel green fluorescence of DCF-DA with the percentage inhibition being relative to A549 fluorescence at 100% (one-way analysis of variance (ANOVA), Tukey's post-test *P≤0.05 in comparison to the control (LPS alone) (n=3));

FIG. 25 is a bar graph showing the effect of Curosurf® at different DPPC concentrations of 500 to 1500 μg/ml on LPS-stimulated oxidative burst measured by mean channel green fluorescence of DCF-DA with the percentage inhibition being relative to A549 fluorescence at 100% (one-way analysis of variance (ANOVA), Tukey's post-test ***P≤0.001 in comparison to the control (LPS alone) (n=3));

FIG. 26 is a bar graph showing the effect of Liposurf® at different DPPC concentrations of 500 to 1500 μg/ml on LPS-stimulated oxidative burst measured by mean channel green fluorescence of DCF-DA with the percentage inhibition being relative to A549 fluorescence at 100% (one-way analysis of variance (ANOVA), Tukey's post-test *P≤0.05 in comparison to the control (LPS alone) (n=3));

FIG. 27 is a bar graph showing the effect of Synsurf® at different DPPC concentrations of 500 to 1500 μg/ml on LPS-stimulated oxidative burst measured by mean channel green fluorescence of DCF-DA with the percentage inhibition being relative to A549 fluorescence at 100% (one-way analysis of variance (ANOVA), Tukey's post-test **P≤0.01 in comparison to the control (LPS alone) (n=3));

FIG. 28 is a set of SEM images labelled A to G showing the stimulation of Actin

Structure Formation and Polymerization in LPS stimulate Rat Alveolar Marchophage (A) Control (B) 1 μg/ml LPS Stimulated (C and G) Synsurf® 1500 μg/ml, 24 h (D) Curosurf® 1500 μg/ml, 24 h (E) Curosurf® 1500 μg/ml, 24 h Phase-Contrast (F) Liposurf® 1500 μg/ml, 24 h;

FIG. 29 is a schematic diagram showing the protein-protein interaction (PPI) network visualised by STRING for Synsurf® exposed LPS-stimulated AMs with only the associated proteins shown;

FIG. 30 is a plot of the trans-epithelial electrical resistance in Calu-3 cells at ALI;

FIG. 31 is a plot of the overall permeability coefficients (Pₐₚₚ) values measured of Linezolid, Linezolid Prep 1 and Linezolid Prep 2 across the Calu-3 transwell in Stage 2;

FIG. 32 is a plot of the overall permeability coefficients (Pₐₚₚ) values measured of Linezolid, Linezolid Prep 1 and Linezolid Prep 2 across the Calu-3 transwell in Stage 3;

FIG. 33 is a plot of the overall permeability coefficients (Pₐₚₚ) values measured of Linezolid, Linezolid Prep 1 and Linezolid Prep 2 across the Calu-3 transwell in Stage 4;

FIG. 34 is a bar graph showing the remaining concentration values measured for Linezolid, Linezolid Prep 1 and Linezolid Prep 2 on the Calu-3 cells in Stage 2;

FIG. 35 is a bar graph showing the remaining concentration values measured for Linezolid, Linezolid Prep 1 and Linezolid Prep 2 on the Calu-3 cells in Stage 3;

FIG. 36 is a bar graph showing the remaining concentration values measured for Linezolid, Linezolid Prep 1 and Linezolid Prep 2 on the Calu-3 cells in Stage 4;

FIG. 37 is a bar graph showing the remaining concentration values measured for Linezolid, Linezolid Prep 1 and Linezolid Prep 2 within the Calu-3 cell lysates in Stage 2;

FIG. 38 is a bar graph showing the remaining concentration values measured for Linezolid, Linezolid Prep 1 and Linezolid Prep 2 within the Calu-3 cell lysates in Stage 3;

FIG. 39 is a bar graph showing the remaining concentration values measured for Linezolid, Linezolid Prep 1 and Linezolid Prep 2 within the Calu-3 cell lysates in Stage 4;

FIG. 40 is a bar graph showing the permeability coefficients (Pₐₚₚ) values measured of Linezolid, Linezolid Prep 1 and Linezolid Prep 2 across the Calu-3 transwell in Stage 2 at 20 minutes;

FIG. 41 is a bar graph showing The permeability coefficients (Pₐₚₚ) values measured of Linezolid, Linezolid Prep 1 and Linezolid Prep 2 across the Calu-3 transwell in Stage 4 at 20 minutes;

FIG. 42 is a set of SEM images of Calu-3 epithelial layers grown at ALI where cilia on the surface is visible as well as a mucosal layer;

FIG. 43 is a SEM image showing (A) Linezolid particles deposited on top of the cells for Stage 2; and (B) examples of tight junction belt fractures after freeze-drying for SEM;

FIG. 44 is a set of SEM images visualising the deposition of Synsurf® and Linezolid on the Calu-3 epithelial layers grown at ALI immediately post pressurised meter dose inhaler (pMDI)-fire; and

FIG. 45 is a set of SEM images visualising the deposition of Synsurf® on the Calu-3 epithelial layers grown at ALI. Unique spreading properties over the mucosal layers are visible 60 seconds post pMDI-fire for (A & B) Linezolid+Prep 1 and (C) Linezolid+Prep 2.

### DETAILED DESCRIPTION WITH REFERENCE TO THE DRAWINGS

A synthetic pulmonary surfactant composition for use as an active component for the treatment of inflammatory and/or cell proliferative disorders of the lungs is provided. The synthetic pulmonary surfactant composition, also referred to herein as “Synsurf®”, has been described in the applicant's International Application number PCT/IB2011/000394 (PCT publication number WO2011/104621), which is incorporated by reference herein. The synthetic surfactant composition consists of a lipidaceous carrier and a peptide complex of poly-L-lysine or a pharmaceutically acceptable salt thereof and poly-L-glutamic acid or poly-L-aspartic acid or a pharmaceutically acceptable salt thereof, the peptide complex having a charge-neutralised region and a positively-charged region.

The poly-L-lysine or salt thereof is generally longer than the poly-L-glutamic acid or poly-L-aspartic acid or salt thereof by at least 17 residues, by at least 35 residues, by at least 50 residues or by at least 85 residues. For example, the poly-L-lysine or salt thereof may be longer by about17 to 49 residues, about 50 to 85 residues or about 35 to 67 residues. As the poly-L-lysine is predominantly positively charged and the poly-L-glutamic acid is predominantly negatively charged, the peptide complex that forms between these two polypeptides has an essentially charge-neutralised region and an essentially positively-charged region. The charge-neutralised region of the peptide complex is capable of interacting with the lipidaceous carrier, while the positively-charged region is available to interact with an aqueous and/or polar environment.

The ratio of the first polypeptide to the second polypeptide is about 1:0.3 (w/w); and the ratio of the peptide complex to the lipidaceous carrier is about 3:100 (w/w).

The poly-L-lysine is typically in the form of poly-L-lysine.HBr, having the formula (I) where n is from about 100 to about 135, more preferably from about 103 to about 135, and even more preferably from about 103 to about 119.

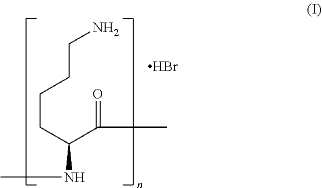

The poly-L-glutamic acid is typically in the form of poly-L-glutamic acid sodium salt, having the formula (II) where x is at least 50, more preferably at least 68 or even more preferably at least 86.

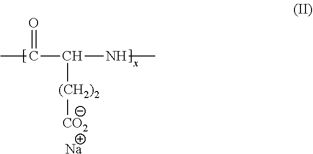

The lipidaceous carrier can include one or more of dipalmitoyl phosphatidylcholine (DPPC), dipalmitoyl phosphatidylglycerol (PG), hexadecanol, cholesterol, tyloxapol or sodium chloride. The ratio of the DPPC, hexadecanol and the PG can be about 10:1.1:1 (w/w).

The composition can optionally include cholesterol; for example from about 3 mg/ml to about 4.8 mg/ml, so at to comprise from about 5 to about 8% (w/w) of the composition.

One example of a suitable synthetic pulmonary composition is a composition which comprises:

| | | |
|---|---|---|
| dipalmitoyl phosphatidylcholine (DPPC) | 60 | mg/ml; |
| dipalmitoyl phosphatidylglycerol (PG) | 6 | mg/ml; |
| hexadecanol | 6.7 | mg/ml; |
| tyloxapol | 1 | mg/ml; |
| poly-L-lysine•HBr | 1.98 | mg/ml; |
| poly-L-glutamic acid sodium salt | 0.613 | mg/ml; and |
| sodium chloride | 100 | mM. |

The poly-L-lysine (or salt thereof) and poly-L-glutamic acid (or salt thereof) were added to the phospholipids in order to mimic the hydrophobic and hydrophilic nature of the naturally occurring pulmonary proteins SP-B or SP-C in the mixture.

As used herein, the term “lipidaceous carrier” means a mixture of phospholipids and optionally other lipid components, for example neutral lipids such as triacylglycerols, free fatty acids and/or cholesterol.

As used herein, the terms “comprising predominantly of” or “essentially” of mean to comprise mainly of. For example, a region having a predominantly or essentially positive charge means that the overall (or net) charge of the region is positive.

As used herein, the term “dipalmitoylphosphatidyl choline” refers to 1,2-Dihexadecanoyl-sn-glycero-3-phosphocholine.

As used herein, the term “phosphatidylglycerol” refers to 1,2-Diacyl-sn-glycero-3-phospho-[1-rac-glycerol].

As used herein, “an effective dose”, “therapeutic dose” or “therapeutically effective amount” for treating a disease is an amount that is sufficient to ameliorate, or in some manner reduce the symptoms associated with the disease. The amount will depend on the kind and the severity of the disease, the characteristics (weight, sex, age) of the subject and the route of administration.

As used herein, the phrase “inflammatory disorder of the lungs” refers to any disorder, disease or infection which results in an inflammatory response in lung cells, including chronic diseases that result in ongoing inflammatory processes in the lung cells and lung infections with microbes which may or may not be associated with such chronic diseases.

As used herein, the term “cell proliferation disorder of the lungs” refers to any growth or tumour caused by abnormal or uncontrolled cell division in the lungs and includes solid tumours such as lung carcinomas and lung adenocarcinomas.

As used herein, the term “lung infection” refers to an infection of the lung arising from the presence of microorganisms or microbes such a bacteria, fungi, or viruses in the lungs.

As used herein, the terms “co-administered” or “co-administration” refers to administration of an active agent or ingredient as an adjuvant therapy to the administration of the surfactant composition. It includes the administration of the active agent or ingredient in combination with the surfactant composition as described below, but is not limited thereto. In certain therapies, the active agent or ingredient may be administered during the entire course of administration of the surfactant composition, may be administered for a period of time that overlaps with the administration of the surfactant composition or may be administered for a period of time that does not overlap with the administration of the surfactant composition, either before or after its administration. Co-administration of the active agent or ingredient may be administration in the same or different formulations, by the same or different routes and in the same or different dosage form type.

As used herein, the term “combination” when referring to the administration of a surfactant composition and drug or active agent combination means that the drug or active agent is formulated with or otherwise administered in combination with the surfactant composition as an adjuvant therapy to the administration of the surfactant composition via the same route.

The synthetic pulmonary surfactant composition may be co-administered together with one or more further active ingredients or drugs. The surfactant composition may be administered in combination with the one or more further active ingredients or drugs. Preferably, the surfactant composition forms part of an inhalable composition including the one or more further active ingredients or drugs. The surfactant composition itself is an active component as either an anti-inflammatory or an anti-cell proliferation agent for treating inflammatory and/or cell proliferation disorders such as cancer in the lungs.

The surfactant composition modulates cell metabolism and the intracellular pathways involved in the inflammatory response and cell proliferation so that it can be used in the treatment of inflammation or cell proliferation disorders. The modulation plays a role in the induction of the respiratory burst, resulting in the production of reactive oxygen species (ROS) which, in turn, induce cell death. Apoptosis is a type of cell extinction regulated in an orderly manner by a series of signal cascades in certain situations, and it is an important physical process involved in regulating growth, development, and immune responses. The induction of apoptosis in tumour cells with the synthetic surfactant (preferably alongside other treatments) results in the synthetic surfactant finding use in therapy for cancer and immune system diseases that affect the pulmonary system.

It has been found that the surfactant composition reduces the inflammatory response and cell proliferation by inhibiting the secretion of pro-inflammatory cytokines and chemokines by alveolar macrophages. The inhibition of the secretion of the pro-inflammatory cytokines and chemokines such as TNF-α, IL-1β, IL-6 and KC/GRO is associated the modulation of intracellular pathways involved with the repair of oxidative stress. For example, it was found that Peroxidoxin-1 (Prx1), Thioredoxin 1 (Trx1), Ferritin Heavy Chain (FHC) and the apoptosis regulator BAX are upregulated in alveolar macrophages in the presence of the surfactant composition. The surfactant composition can therefore be used to treat inflammation or cell proliferation disorders in the lungs when a therapeutically effective amount is administered to the lungs via intubation, direct pulmonary administration or inhalation. It is preferred that the surfactant composition is administered via the less invasive method, i.e. inhalation. In order to make the surfactant composition inhalable it must be in an inhalable formulation which optionally includes one or more of a carrier, a propellant, a solubiliser, a preservative and/or a stabiliser and may be formulated for nebulisation, to form a liquid aerosol or to be in the form of an inhalable powder. Any suitable inhaler may be used to administer the inhalable formulation, such as a pressurised meter dose inhaler, a nebuliser or a dry-powder inhaler. More generally, the surfactant composition may be used in the manufacture of a medicament for the treatment of inflammation or cell proliferation disorders.

The threshold concentration for treating inflammation or cell proliferation disorders of the lungs with the synthetic pulmonary surfactant compositions is a DPPC concentration of 500 μg/ml.

The synthetic pulmonary surfactant composition may be used in the treatment of cell proliferation disorders of the lungs such as lung cancer, particularly a lung carcinoma, more particularly a lung adenocarcinoma. The development and chronicity of cancers has been attributed to the chronic inflammation in affected organs such as the lungs. In order to treat the cancer, a therapeutically effective amount of the surfactant composition is administered to the lungs via intubation, direct pulmonary administration or inhalation, preferably by the less invasive method of inhalation in which case the surfactant is in an inhalable formulation. A medicament in the form of an inhalable formulation may be manufactured using the surfactant composition for cancer treatment. Again, a pressurised meter dose inhaler, nebuliser or dry-powder inhaler may be used to administer a therapeutically effective amount of the surfactant composition to treat lung cancer. The therapeutically effective amount of the surfactant composition may depend on the dosage (including the phospholipid concentration) of the surfactant composition and the exposure time of the cancer to the surfactant composition. It has been found that a threshold concentration (effective concentration) of the surfactant composition Synsurf® is a phospholipid (DPPC) concentration of 500 μg/ml or more when the exposure time is more than 1 hour.

The surfactant composition may include a second or further active component which may be a pharmaceutically active composition or compound which actively treats a disease characterised by inflammation and/or uncontrolled cell proliferation in the lungs. In this regard the surfactant composition may enhance the permeability of the pharmaceutical composition or compound through cell membranes. The surfactant composition may include a chemotherapeutic agent or an antimicrobial such as an antibiotic for example.

In this manner, the synthetic pulmonary surfactant composition may be used as a drug delivery agent in terms of which a synthetic pulmonary surfactant composition and drug combination may provide dual immunomodulatory and/or anti-cancer effects. The surfactant composition and drug may be delivered into the lungs leading to site-specific drug delivery. The delivery preferably occurs by inhalation when the surfactant composition and drug is in an inhalable formulation. The drug is an active component in the form of a pharmaceutical compound or composition. The surfactant composition is also an active component in that it modulates the immune response and/or cell proliferation whilst also acting as a permeabilising agent of cell membranes that increases permeability of the pharmaceutical compound or composition across the membrane.

The combination of the surfactant composition and drug provides for a dual drug delivery system. The polypeptides in the surfactant composition and/or the lipidaceous carrier plays an active role in increasing the efficacy of the drugs in a site-specific manner.

The surfactant composition and drug combination may be used in the treatment of a selected disease when a therapeutically effective amount of the surfactant composition and drug combination is administered to the lungs via intubation, direct pulmonary administration or inhalation. It is preferred for administration to occur via inhalation when it is in an inhalable formulation, as this is less invasive and more accessible to patients suffering from a disease. The surfactant composition and drug combination may be used in the manufacture of a medicament, preferably an inhalable composition, for the treatment of a disease.

Diseases that may be treated in this manner include, for example, chronic obstructive pulmonary disease (COPD) and respiratory distress syndrome (RDS) by delivery of active components such as Isoniazid (INH), Moxifloxacin, Ofloxacin, Pyrazinamide, and/or Linezolid. Cystic fibrosis can be treated by administration of a surfactant composition in combination with Tobramycin for example. Whilst pneumonia or similar bacterial infections may be treated using amoxicillin and ceftazidime for example.

An infection of the lungs by Gram positive bacteria such as a Mycobacterium tuberculosis infection (TB) can be treated by the administration of the synthetic surfactant composition and an antibiotic combination. The antibiotic may be from the oxazolidinone class such as Linezolid. The surfactant composition and Linezolid combination can be used in the treatment of a Mycobacterium tuberculosis infection (TB).

Traditionally, exogenous surfactants have been administered by tracheal instillation or intubation, intra-tracheal catheters, bronchoscopies and laryngeal mask airway devices. Nebulization and inhalation poses a less invasive method of administration of exogenous surfactants. Consequently, surfactant compositions are being considered for wider use in the treatment of diseases and disorders and their associated symptoms, provided that no inflammatory response results following its administration. When the synthetic surfactant composition was tested for an inflammatory response to elucidate its potential safety for use as a natural surfactant replacement in preterm infants, it was surprisingly found that the surfactant composition had an anti-inflammatory effect at a threshold concentration. This led to the testing of other commercially available animal-derived surfactant compositions (Exosurf® and Liposurf®) in addition to Synsurf®, an embodiment of the synthetic surfactant composition, to compare their respective anti-inflammatory effects. The tests revealed the immunogenicity and immunomodulatory functions of Synsurf®.

### EXAMPLES

The following examples illustrate and exemplify how the synthetic surfactant composition can be used in the treatment of inflammation or cell proliferation disorders such as cancer, and as a drug delivery agent with dual immunomodulatory effects. The activity of the synthetic surfactant composition is compared to that of commercially available animal-derived exogenous pulmonary surfactants, Curosurf® and Liposurf®.

### Example 1—The Inhibition of Pro-Inflammatory Cytokines

The lungs present an immunological challenge for the host as they are most frequently targeted by pathogens. The respiratory epithelium serves as the surface for gaseous exchange along with its ability to fight inhaled pathogens, pollutants and particles. The epithelium represents a physical barrier that produces mucus, which, along with, mucociliary clearance combats any possible onslaught.

A variety of cells participate in the pulmonary immune response, and the relative involvement of different cells varies with different diseases and the degree of inflammation. The innate immunity is the first line of defence and it recruits a number of leukocytes including basophils, eosinophils, natural killer (NK) cells, mast cells along with the phagocytic cells macrophages, neutrophils and dendritic cells. The immune cells recognise the pathogen by relying on a large family of pattern recognition receptors (PRRs) termed pathogen-associated molecular patterns (PAMPs). These immune cells can either engage in phagocytosis to “engulf” pathogens and release inflammatory mediators such as histamine and leukotrines (eicosanoid inflammatory mediators).

The adaptive immune system is also known as the acquired immune system and it is composed of highly specialized, systemic cells. These cells create an immunological memory when they encounter a pathogen, with a specific response, which will be enhanced in subsequent encounters with that pathogen. The adaptive immune response involves dendritic cells and T and

B lymphocytes (and to a lesser extent macrophages) which are known as professional antigen presenting cells.

Inflammation is a self-protective mechanism initiated by the body's innate immune system against tissue injury or infection, but may elicit an adaptive trait by recruiting cells associated with the system to act in collaboration. It underlines a wide variety of physiological and pathological processes. In the pathological aspect, it restricts the tissue damage at an affected site and is characterized by the secretion of numerous inflammatory mediators, which trigger the recruitment of leukocytes and other proteins. Much progress has been made in understanding the cellular and molecular mechanisms found in the acute inflammatory response to infection and, to a lesser extent, tissue injury. Additionally, the events leading up to localised chronic inflammation, particularly that which is associated in chronic infections and autoimmune diseases, are only partially known. Systemic chronic inflammation's causes and mechanisms are much less known but do not seem to be caused by the same activators as those associated with infection and injury. It is rather accompanied with the malfunction of tissue. That said, it is the homeostatic imbalance of one of several physiological systems that are not directly related to the function of host defence or tissue repair. The fundamental function of the inflammatory process is to resolve the infection and repair the damage tissue to return the system back to a state of homeostasis. The effectiveness of this function relies on the system's ability to produce a rapid response whilst ideally limiting the damage to a specific area rapidly. However, chronic inflammation, as seen in COPD, causes more damage to the system than a microbe trigger.

The innate immune response is the first line of defence against an invading pathogen or infection and is activated within a few hours of exposure. The strategy that it employs has been termed “pattern recognition” using “pattern-recognition receptors” (PRRs) that recognise pathogen-associated molecular patterns (PAMPS). Lipopolysaccharides (LPS) and peptidoglycans from bacterial cell walls are examples of such patterns. Alongside neutrophils and DCs, Macrophages, which link the innate and adaptive immune systems, possess a type of germline-encoded receptor/PRR called Toll-like receptors (TLRs) on their surface and within their cytoplasm. Once the TLRs detect PAMPs, the innate immune system is initiated and the release of pro-inflammatory mediators such as cytokines, chemokines and lipid mediators into the circulation is initiated. It does so in distinctive dynamic pattern and results in the activation, recruitment and/or migration of cells to the site of infection.

The IL-1 receptor (IL-1 R) family members consist of very wide related receptors, including the TLRs, and are characterised by very different extracellular immunoglobulin-like domains and large intracellular Toll/IL-1 R (TIR) domains. When the TLRs are activated, adapter proteins and kinases, such as MyD88 and IRAK, are recruited.

The transcription factors AP-1, CREB, NF-κB and IRF3 are activated through direct or indirect mechanisms after which they are translocated to the nucleus. There, they induce gene transcription of pro-inflammatory factors. The wide variety of target genes regulated by NF-κB includes those encoding cytokines e.g. IL-1, IL-2, IL-6, IL-12, TNF-α, LTα, LTα, and GM-CSF and chemokines e.g., IL-8, MIP-1α, MCP1, RANTES, CXCL1/KC and eotaxin. IL-1 and TNF-α are exploited in a feed forward loop to initiate and intensify the inflammatory response by re-binding to the IL-1Rs. The inflammatory response is driven and maintained by highly integrated gene transcription control within the nucleus of the cell, as well as with multiple steps of post-transcriptional modifiers that modulate mRNA production, transport, translation and posttranscriptional regulation. The rates of mRNA transport, decay, and translation crucially influences the timing and magnitude of the cellular immune responses.

Natural pulmonary surfactant serves two functions in the lung. Firstly, it is a surface acting agent, initially identified as a lipoprotein complex that lowers surface tension at the air-liquid interface of the alveolar surface. Secondly, the hydrophilic surfactant proteins SP-A and SP-D (also known as collectins) are important components of the innate immune response in the lung and therefore assist in pulmonary host defence. They may also modulate the adaptive immune response. The inflammatory response in the alveolar microenvironment is tightly regulated to avoid damage to the gas-exchanging delicate structures through the concerted efforts of the innate and adaptive immune system.

The phospholipid and protein combination has unique spreading qualities (90% phospholipids and 10% proteins) courtesy of the hydrophobic surfactant proteins B and C (SP-B, SP-C). This promotes lung expansion during inspiration and prevents lung collapse during expiration. They have an essential function in the spreading, adsorption and stability of surfactant lipids. Surfactant composition and pool size is controlled by secretion, re-uptake, and recycling by alveolar type II epithelial cells and both alveolar type II epithelial cells and macrophages are responsible for the degradation thereof.

Harvesting alveolar macrophages (AMs) by bronchoalveolar lavage was first described in 1960 and has since been extensively investigated. They are critical for host defence as they are the major cell type in recognising infection or injury and innate immunity and comprise 85% of the recovered cells in human lung lavage fluid. They phagocytose particulate matter and invading microorganisms, release cytotoxic reactive oxygen species (ROS) and proteolytic enzymes, and produce nitric oxide (NO) for microbial killing and signalling functions. As previously stated, AMs bring about the pulmonary inflammatory response via production of cytokines and chemokines as they are responsive to both specific and nonspecific stimuli, thereby being capable of forming part of both the innate and adaptive immunity. Furthermore they regulate antigen presentation and opsonisation. AMs also remove intra-alveolar debris whilst regulating the metabolism and recycling of endogenous surfactant.

There is a need for the development and testing of exogenous surfactant compositions that have immunoactive properties. Long-term surfactant exposed AMs may elicit a basal inflammatory phenotype whilst surfactant may, in contrast, act as a protective buffer to LPS stimulated AMs by inhibiting the production of pro-inflammatory cytokines. It is therefore also necessary to ascertain a therapeutically effective amount of a surfactant composition that has a threshold “switch” related to surfactant concentration and exposure time to AMs.

### Experimental Section

In this section, the effects of three exogenous surfactants on NR8383 AMs, an immortalized rat alveolar macrophage cell line, is characterized.

### Materials and Methods

DPPC was obtained from Avanti Polar Lipids (Alabaster, Ala., USA). PG, cetyl alcohol, tyloxapol, poly-L-lysine (molecular weight 16.1 kDa) and poly-L-glutamic acid (molecular weight 12 kDa) were purchased from Sigma-Aldrich (St Louis, Mo., USA). Phospholipid purity was verified by thin-layer chromatography. Sterile water for injection was used in the preparation of surfactant. Chloroform used was high-performance liquid chromatography-grade (Merck, Darmstadt, Germany).

### Surfactant Compositions

CUROSURF® is a natural surfactant, prepared from porcine lungs, containing almost exclusively polar lipids, in particular phosphatidylcholine (about 70% of the total phospholipid content), and about 1% of specific low molecular weight hydrophobic proteins SP-B and SP-C.

LIPOSURF® is an extract of natural bovine surfactant which contains numerous phospholipids with dipalmitoylphosphatidylcholine (DPPC) being the most abundant. It also includes hydrophobic surfactant-associated proteins SP-B and SP-C.

SYNSURF® is a synthetic peptide-containing surfactant consisting of phospholipids (DPPC being the most dominant, ≅80%) and poly-L-lysine electrostatically bonded to poly-L-glutamic acid.

An embodiment of Synsurf® used in the below examples was prepared by mixing DPPC, hexadecanol, and PG in a 10:1.1:1 ratio (w/w) in chloroform. The organic solvent was then removed by rotary evaporation and the mixture was dried under a continuous stream of nitrogen at room temperature. Poly-L-lysine (-100-120 residues) was mixed with poly-L-glutamate (approximately 80 residues) and incubated at 3° C. in 0.1 M NaCl to give a complex that was about 50% neutralized. The complex was prepared in such a manner as to be net positively charged through having an excess of poly-L-lysine residues. The dried phospholipid film was then hydrated with the polymer mixture (3% by weight of the phospholipid concentration) and gently mixed in the presence of glass beads. A Branson (Danbury Conn., USA) B-15P ultrasonicater fitted with a microtip was then used to sonicate the mixture on ice under a stream of nitrogen (power of 20 watts for 7×13 seconds; 60-second intervals). Hereafter, 24 mg of tyloxapol was added to the preparation, and the tube was sealed under nitrogen before use.

The composition of Synsurf® used in all of the below examples is as follows:

| | | |
|---|---|---|
| dipalmitoyl phosphatidylcholine (DPPC) | 60 | mg/ml; |
| dipalmitoyl phosphatidylglycerol (PG) | 6 | mg/ml; |
| hexadecanol | 6.7 | mg/ml; |
| tyloxapol | 1 | mg/ml; |
| poly-L-lysine•HBr | 1.98 | mg/ml; |
| poly-L-glutamic acid sodium salt | 0.613 | mg/ml; and |
| sodium chloride | 100 | mM. |

Since DPPC is the main component of Synsurf®, dilution series based on the DPPC concentrations were prepared and used in all the experiments. The dilution series were used to indicate threshold effects.

### Cell Culture

The NR8383 Alveolar Macrophages (AMs) were first cultured in 75 cm² flasks and maintained in a humidified, 5% CO₂-95% atmospheric air incubator at 37° C. The media, comprised of RPMI 1640 (Roswell Park Memorial Institute media) supplemented with 10% fetal calf serum, 1% L-glutamine solution (200 mM), and 1% Penicilin-Streptomycin (PENSTREP), and was routinely changed twice weekly. Cells were seeded to 12-well tissue culture plates at a density of 2.5×10⁵ cells/well. Cell viability before and after each experiment was assessed by trypan blue exclusion. The viability was consistently greater than 95% in all detected samples before seeding as well as after treatment.

### Inflammatory Cytokines

To evaluate the anti-inflammatory effects of exogenous surfactants, Curosurf®, Liposurf® and Synsurf® were standardised to equivalent phospholipid concentrations, 100-1500 μg/ml and incubated with LPS-(1 μg/ml) stimulated and un-stimulated NR8383 AMs over 24 hours. The changes in cytokines were analyzed by using a multiplex (V-PLEX) rat cytokines's electrochemiluminescence-based ELISA kit (Meso Scale Discovery®) as per the manufacturer's instructions. The values were first calculated for picogram cytokine per milliliter (pg/ml) of sample, and then converted into microgram (μg/ml) where relevant. Some samples that had low signals which were below the detection threshold and these were excluded from data analysis.

### Results

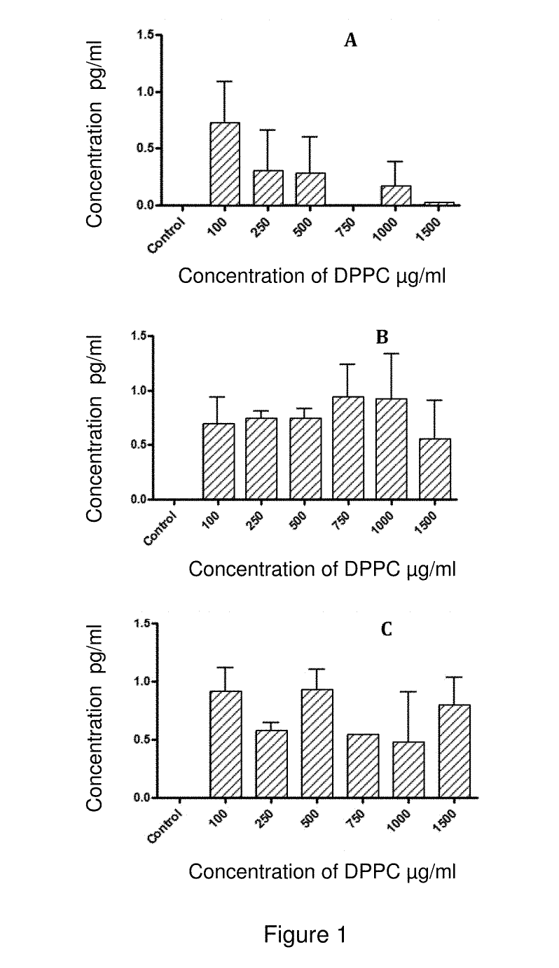

The effects of Curosurf®, Synsurf® and Liposurf® on un-stimulated TNF-α production are shown in FIG. 1. FIG. 1 shows the concentration of TNF-α in cell supernatant by NR8383 AM in the presence of (A) Curosurf®, (B) Synsurf® and (C) Liposurf® at 100-1500 μg/ml phospholipids.

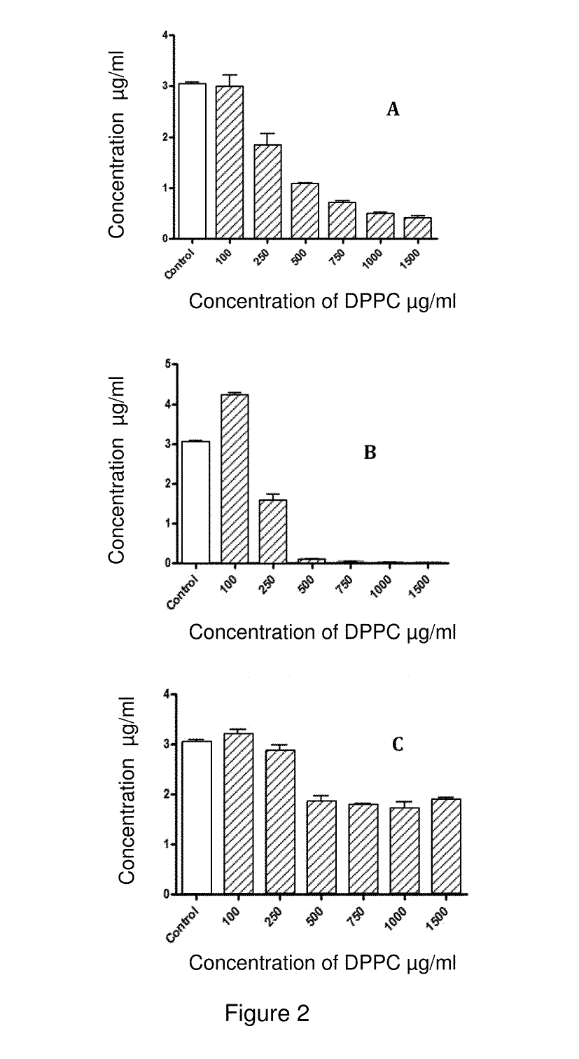

The effects of Curosurf®, Synsurf® and Liposurf® on LPS-stimulated TNF-α production are shown in FIG. 2. FIG. 2 shows the concentration of TNF-α in cell supernatant by LPS-stimulated NR8383 AM in the presence of (A) Curosurf®, (B) Synsurf® and (C) Liposurf® at 100-1500 μg/ml phospholipids.

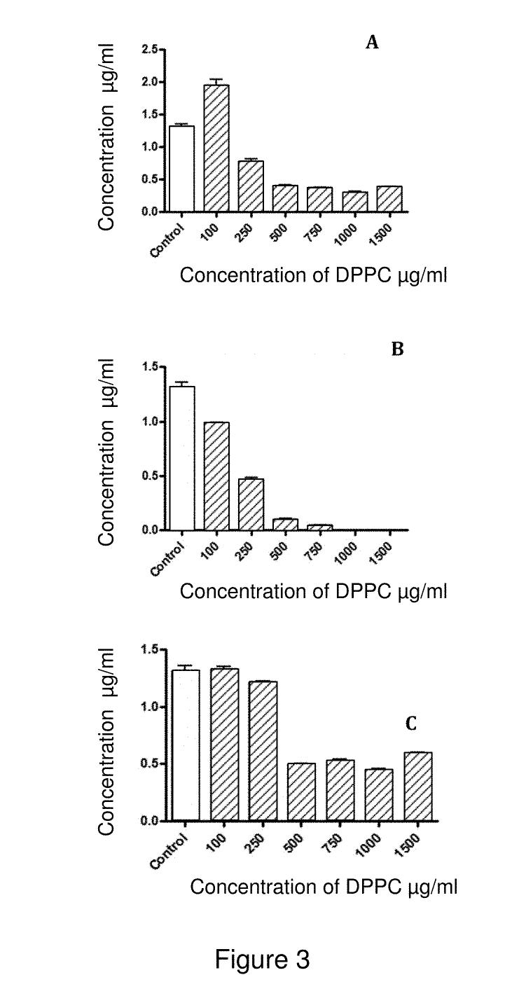

The effects of Curosurf®, Synsurf® and Liposurf® on LPS-stimulated IL-1β production are shown in FIGS. 3. FIG. 3 shows the concentration of IL-1β in cell supernatant by LPS-stimulated NR8383 AM in the presence of (A) Curosurf®, (B) Synsurf® and (C) Liposurf® at 100-1500 μg/ml phospholipids.

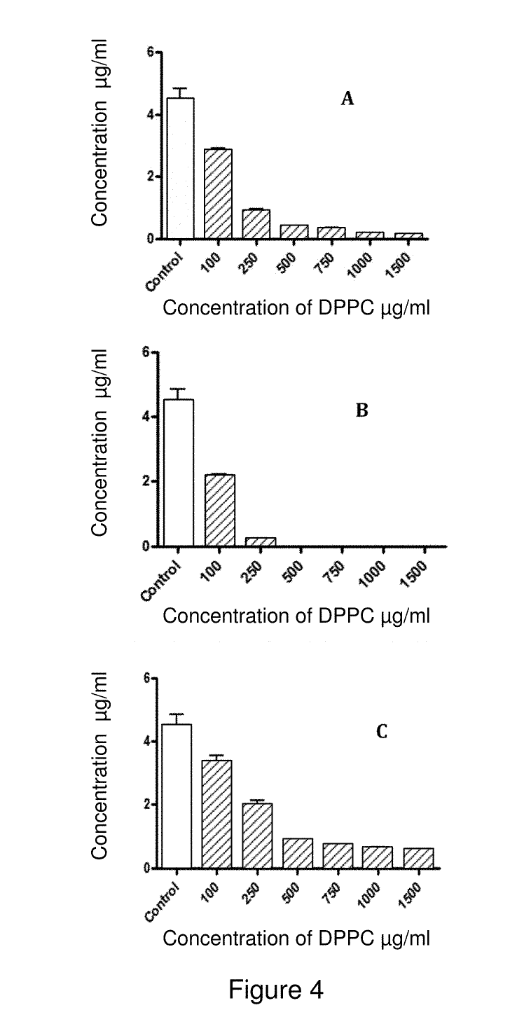

The effects of Curosurf®, Synsurf® and Liposurf® on on LPS-stimulated IL-6 production are shown in FIG. 4. FIG. 4 shows the concentration of IL-6 in cell supernatant by LPS-stimulated NR8383 AM in the presence of (A) Curosurf®, (B) Synsurf® and (C) Liposurf® at 100-1500 μg/ml phospholipids.

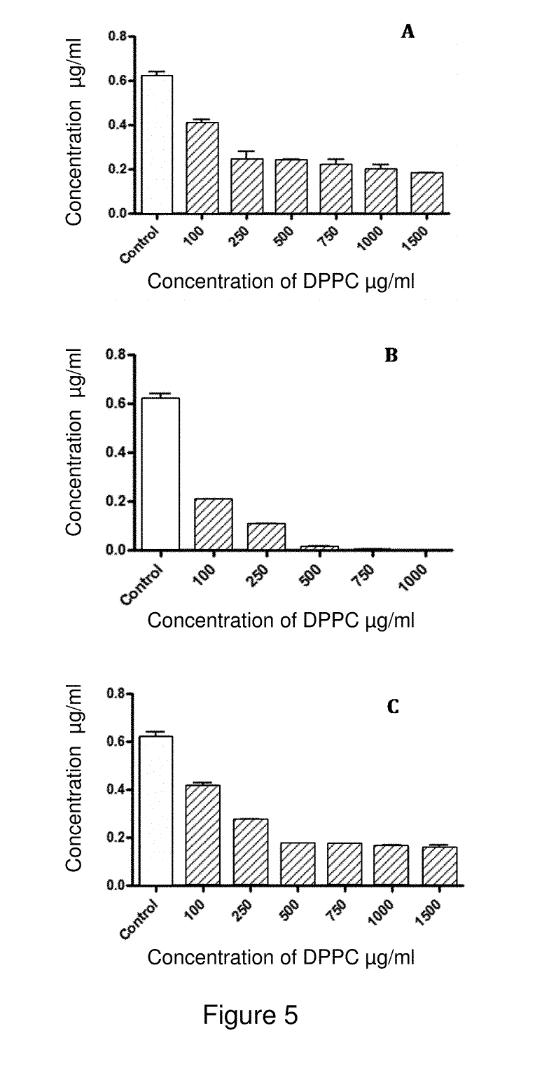

The effects of Curosurf®, Synsurf® and Liposurf® on on LPS-stimulated KC/GRO production are shown in FIG. 5. FIG. 5 shows the concentration of KC/GRO in cell supernatant by LPS-stimulated NR8383 AM in the presence of (A) Curosurf®, (B) Synsurf® and (C) Liposurf® at 100-1500 μg/ml phospholipids.

It was surprisingly found that the synthetic surfactant, Synsurf®, exerts low-level pro-inflammatory effects in alveolar macrophages. Moreover, there is some evidence that in the context of pre-existing lung inflammation, Synsurf® elicited a limiting or reducing effect on inflammation in a dose-dependent manner by decreasing TNF-α, IL-1β, and IL-6 production in alveolar macrophages when in the presence of LPS stimulation thus elucidating a “threshold” concentration characteristic effect. The production of all three pro-inflammatory cytokines and the KC/GRO chemokine, the “threshold—concentration” is displayed at approximately 500 μg/ml phospholipids content after which their production plateaus. The presence of Synsurf® alone did not display a “dose-dependent” low-level TNF-α production or any IL-1β and IL-6 production in the absence of LPS.

When compared to the naturally derived animal derived surfactants Curosurf® and Liposurf®, Synsurf® was more effective in blunting the inflammatory cascade (statistically significant, p<0.05) and it displayed a similar “threshold” potential.

The specific surfactant proteins may be responsible or play a key role for the inflammatory response and the protective mechanisms thereof when AMs are LPS stressed. However, Synsurf®, a synthetic and protein-free surfactant (containing peptide complexes), displayed the same “protective” features, and was even more effective than the animal derived SP-B and/or SP-C containing surfactants. These findings could suggest non-specific lipid protection with AMs as seen by Synsurf® which may or may not be linked to protein content.

As demonstrated in Example 2 below, the surfactant composition (Synsurf®) modulates LPS-mediated cytokine and ROS production via blunting of NF-κB activation in AMs.

### Example 2—In Vitro Studies of the Anti-Inflammatory and Anti-Cancer Effects

Lung cancer-dependent deaths constituted 30% (men) and 26% (women) of the estimated total cancer-related deaths in 2009. Lung cancer patients are in need of new, effective therapeutic options. Since the 1950s, the incidence of lung adenocarcinoma his risen in comparison to other types of lung cancers.

Administration of exogenous lung surfactant can influence the lung epithelial cell surface as well as the agglomeration state of immune cells within the lung. Exogenous surfactant cellular modulation on delicately balanced intracellular pathways may induce a respiratory burst, resulting in the production of reactive oxygen species (ROS) which, in turn, induces cell death. Apoptosis is a type of cell extinction regulated in an orderly manner by a series of signal cascades in certain situations, and it is an important physical process involved in regulating growth, development, and immune responses (FAN, T. J. et al. 2005, Caspase family proteases and apoptosis. Acta Biochimica et Biophysica Sinica, 37, pp. 719-727; CHEN, N.Y et al. 2008, Induction of apoptosis in human lung carcinoma A549 epithelial cells with an ethanol extract of Tremella mesenterica. Bioscience, biotechnology, and biochemistry, 72(5), pp. 1283-1289). The induction of apoptosis in tumour cells with exogenous surfactant (alongside other treatments) might prove to be an important approach in therapy for cancer and immune system diseases that affect the pulmonary system.

Assessing the biological activity of surfactants in cell-based experiments of NR8383 rat alveolar macrophages and A549 alveolar epithelial carcinoma cells may indicate a role of oxidative stress in the production of inflammatory cytokines and cytotoxic cellular responses. The effect of exogenous surfactants on the biogenesis and metabolism as well as morphological changes in rat alveolar macrophages NR8383 and A549 type II respiratory epithelial cells was determined in vitro and thus in the absence of clinical variables. The effects of two animal derived (Curosurf® and Liposurf®) and one synthetic surfactant (Synsurf®) were studied at varying concentrations and time intervals for the use in formulations applied on the respiratory epithelium by means of an in vitro cytotoxicity assay.

### Experimental Section

### Materials and Methods

Surfactant compositions: the same surfactant compositions of Example 1 were used.

### Cell Culture

Both the NR8383 and A549 cell lines were successfully established and viability was established 95-98% before each experiment. Both the NR8383 (Rat Alveolar Macrophages, ATCC®, Cat. No. CRL-2192™) and the A549 (Lung Carcinoma, ATCC®, Cat. No. CCL-185™) cell lines were first cultured in 75 cm² flasks and maintained in a humidified, 5% CO₂-95% atmospheric air incubator at 37° C. The NR8383 cell line media comprised of RPMI 1640 (Roswell Park Memorial Institute media) supplemented with 10% fetal calf serum, 1% I-glutamine solution (200 mM), and 1%

Penicilin-Streptomycin (PENSTREP). The media for the A549 cell line comprised of Advanced DMEM supplemented with 5% fetal calf serum, 1% I-glutamine solution (200 mM), and 1% Penicilin-Streptomycin (PENSTREP), and both were routinely changed twice weekly. Cells were seeded to 48-well tissue culture plates at a density of 2.5×10⁴cells/well for NR8383 and 2×10⁴ cells/well for A549 respectively. Cell viability before each experiment was assessed by trypan blue exclusion.

### Viability Assay

Cell viability was determined by 3-(4,5-Dimethylthiazol-2-yl)-2,5-diphenyltetrazolium bromide (MTT) assay and were performed in triplicate with Curosurf®, Synsurf®, Liposurf® for 30 min, 1, 4, 12 and 24 hrs with final phospholipid (DPPC) concentrations of 25 to 1500 μg/ml for both cell lines. The assay measures the ability of the mitochondria within living cells to reduce the yellow MTT dye to its purple formazan product. This product is then dissolved with isopropanol (1%)/triton (0.1%) solution at a 50:1 ratio; the absorbance reading of the resulting solution is proportional to the number of viable cells. The cells, when established at 80% confluency after seeding, were stimulated according to the specific experimental procedure. At the end of the experiment, the plates containing rat alveolar macrophages were spun down and the supernatants were carefully removed to ensure the collection of all the semi-adherent cells. For the adherent A549 epithelial cell line, the media only needed to be removed. The cells were then incubated (covered in foil) in 250 μl of 2.5 mg/ml MTT solution in PBS for 2 hours in a humidified, CO₂ 5%-95% atmospheric air incubator at 37° C. Confirmation of the purple product within the cells was observed using a light microscope, 250 μl of isopropanol (1%)/triton (0.1%) solution was added to each well to lyse cells and dissolve the formazan product. The plates were placed on a plate shaker for a few minutes to ensure the product is dissolved. The absorbance at 550 nm was measured using a universal microplate reader, EL800 BioTek Instruments Inc. The effect of treatment on cell viability was calculated as a percentage of OD relative to the untreated control.

### Oxidative Burst

The reactive oxidative intermediate, ROS production, in A549 epithelial cells and AMs was measured by flow cytometry. The respective cell lines were treated in culture with Curosurf®, Liposurf® and Synsurf® (500-1500 μg/ml DPPC) for 12 and 24 hours then washed, re-suspended and loaded with the fluorescent probe 2′,7′-dichlorofluorescein acetate (DCFH-DA, 25 μM) (Sigma Aldrich). Esterase cleaves the acetate groups of DFH-DA, thus the trapped DCFH is converted to the highly fluorescent 2′,7′-dichlorofluorescein (DCF) in the presence of reactive oxygen intermediates. DCFH loaded cells were used as the baseline to measure autofluorescence. The fluorescence of cells was recorded under 488 nm excitation and green fluorescence from DCF was measured through a 520 nm band pass filter with a 520 nm dichromic mirror. Fluorescence values from cells loaded with DCFH without surfactant treatment were standardized at 100%. Scattering properties and DCF fluorescence were analysed by FAC-Scan flow cytometer (FACS Calibur, Becton Dickinson). All experiments were repeated three times.

### Microscopic Analysis of F-Actin Structures of NR8383

Microscopic analysis of F-actin structures of LPS stimulated NR8383 was completed for post-24 h treatment with Curosurf®, Synsurf®, Liposurf® in comparison to control and LPS stimulated control (1 μg/ml). Visualization of potential actin polymerization after exposure to surfactants (1) Curosurf®, Synsurf®, Liposurf® with final phospholipid concentrations 750 and 1500 μg/ml DPPC (2) LPS at 1 μg/ml (3) Control-Medium for 24 h. Alveolar Macrophages were allowed to adhere to eight-well glass cell culture slides overnight and non-adherent cells were removed before treatment.

### Staining Procedure

Cells were washed with warm PBS and fixed with 4% paraformadehyde for 10 min and washed 3x thereafter for 5 min with PBS. Cells were incubated with 0.1% triton X-100 for 6 min at 37° C. and thereafter washed 3× for 5 min with PBS. Cells were stained with Phalloidin TRITC (P1951) at 50 μg/ml for 40 min at room temperature and washed 3× for 5 min with PBS. Slides were allowed to air-dry and cells examined using the Zeiss LSM780 ELYRA PS1 confocal microscope.

### Results

### Viability Assays

1. NR8383 Rat Alveolar Macrophages

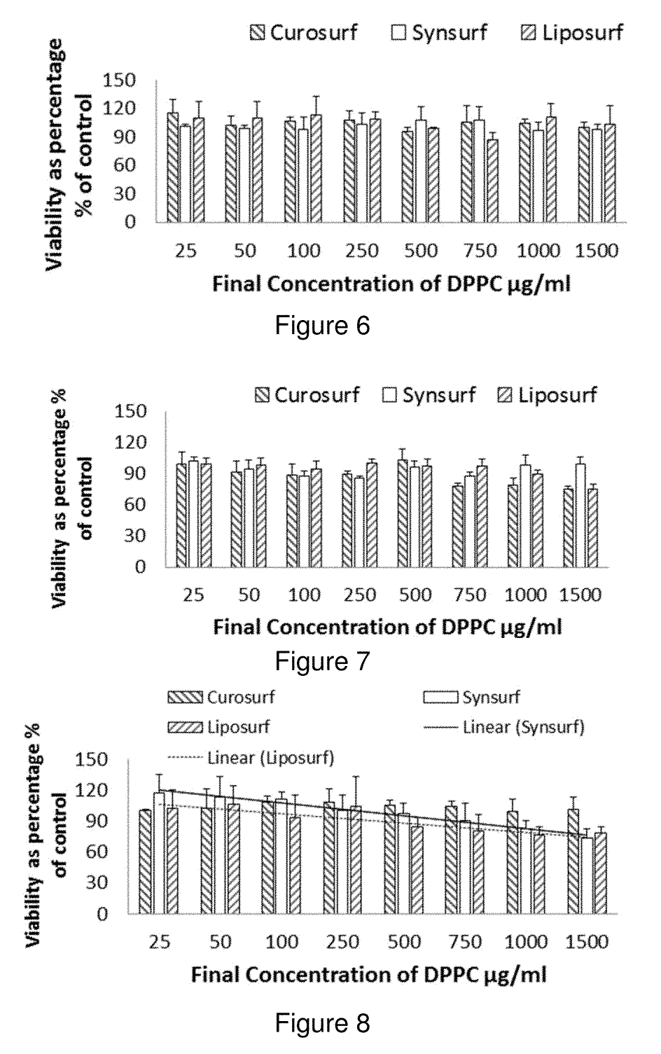

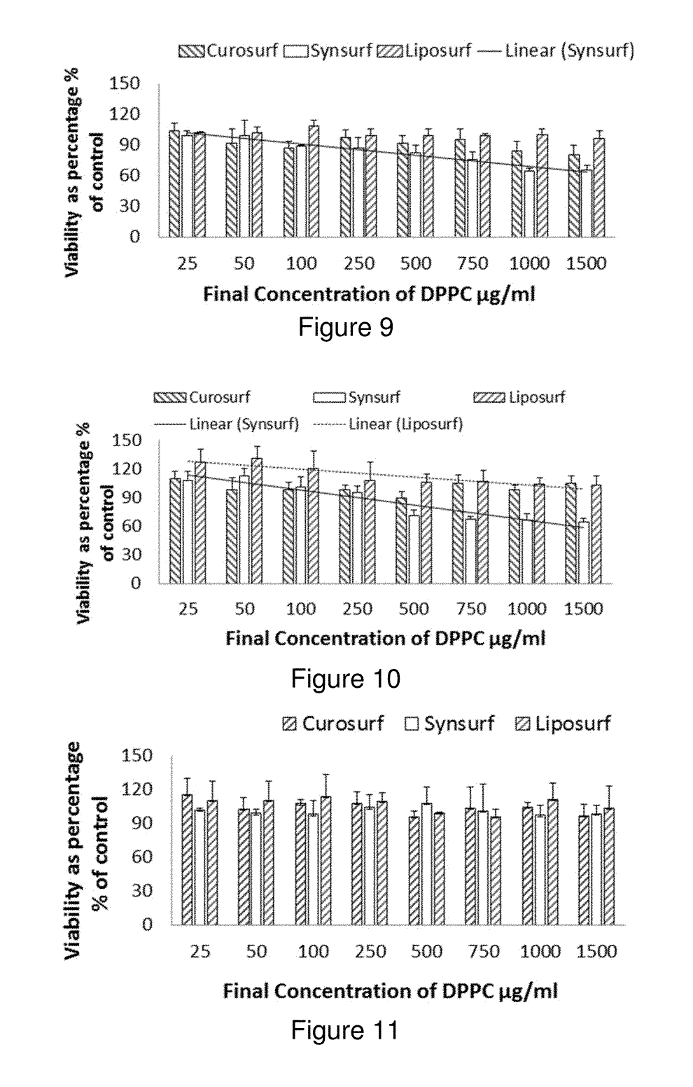

The well-established and commonly used cytotoxicity assay, MTT, was utilized to determine the dose and time dependent toxic effect of three pulmonary surfactants: Curosurf®, Synsurf® and Liposurf® to NR8383 alveolar macrophages. FIGS. 6 to 10 show the plots of the cytotoxic response. The results show dose-dependent as well as time (exposure) dependent cytotoxicity of the surfactants. Decrease in cell viability by exposure to higher phospholipid concentrations of surfactants were only witnessed from 4 h onwards (FIG. 8). Both Synsurf® and Liposurf® displayed a similar trend in dose-dependent cytotoxicity, however, the decrease in cell viability was not deemed significant. Synsurf® exhibited the same trend for the 12 h (FIGS. 9) and 24 h (FIG. 10) time exposure and significant decreases in cell viability were seen at phospholipid concentrations of 1000 μg/ml (***P≤0.001) and 1500 μg/ml (**P≤0.01) for 12 h and again 1000 μg/ml (*P≤0.05) and 1500 μg/ml (*P≤0.05) for 24 h.

2. A549 Lung Carcinoma

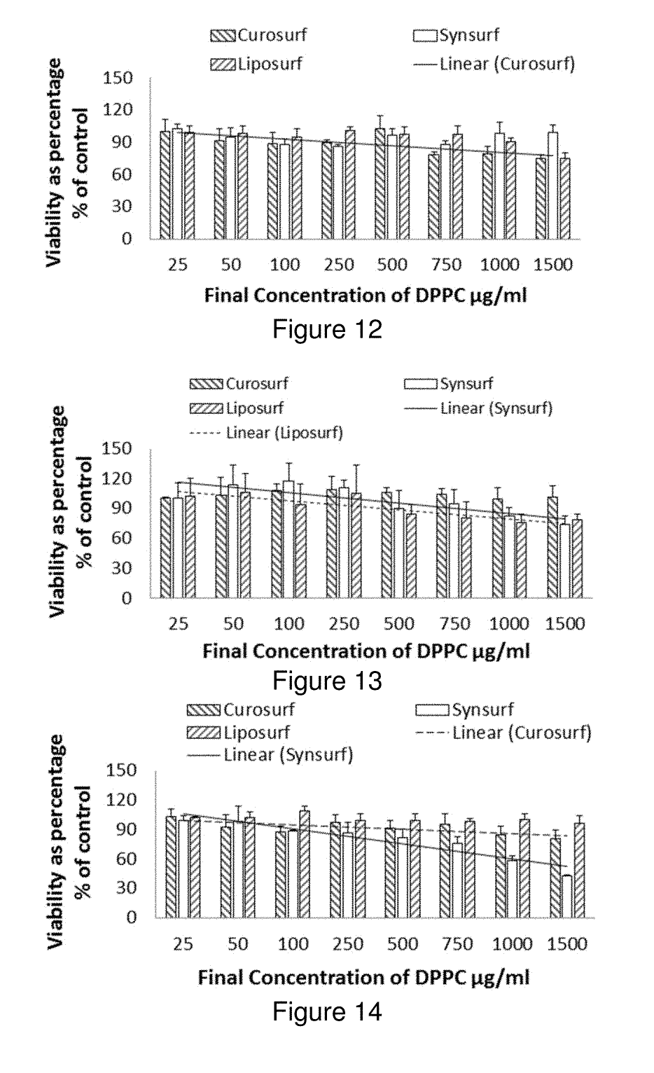

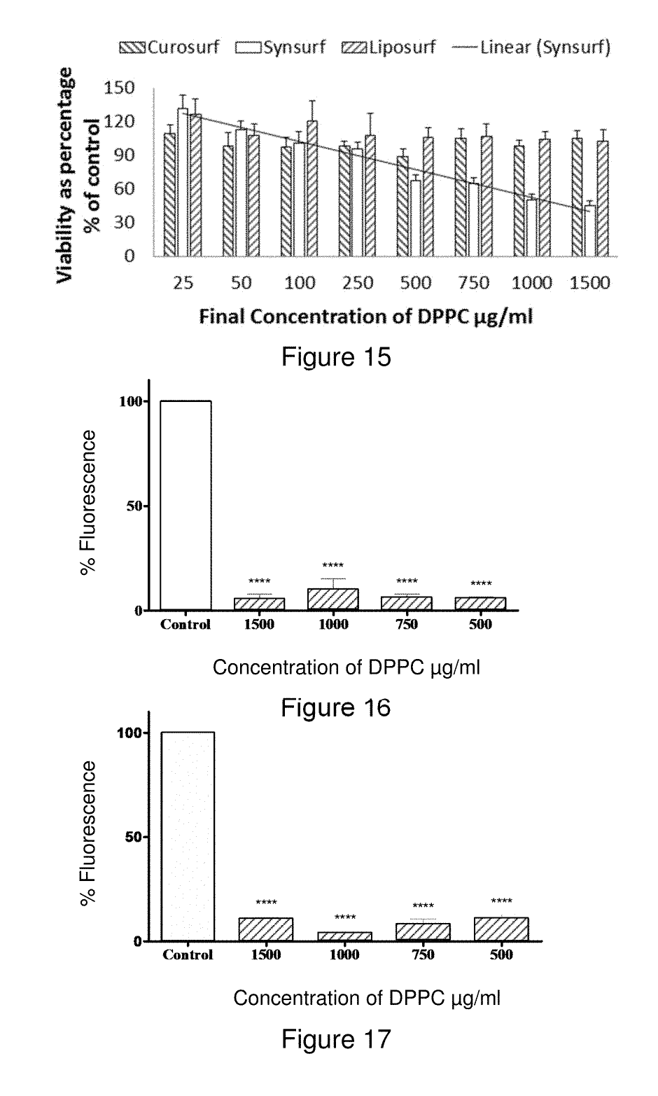

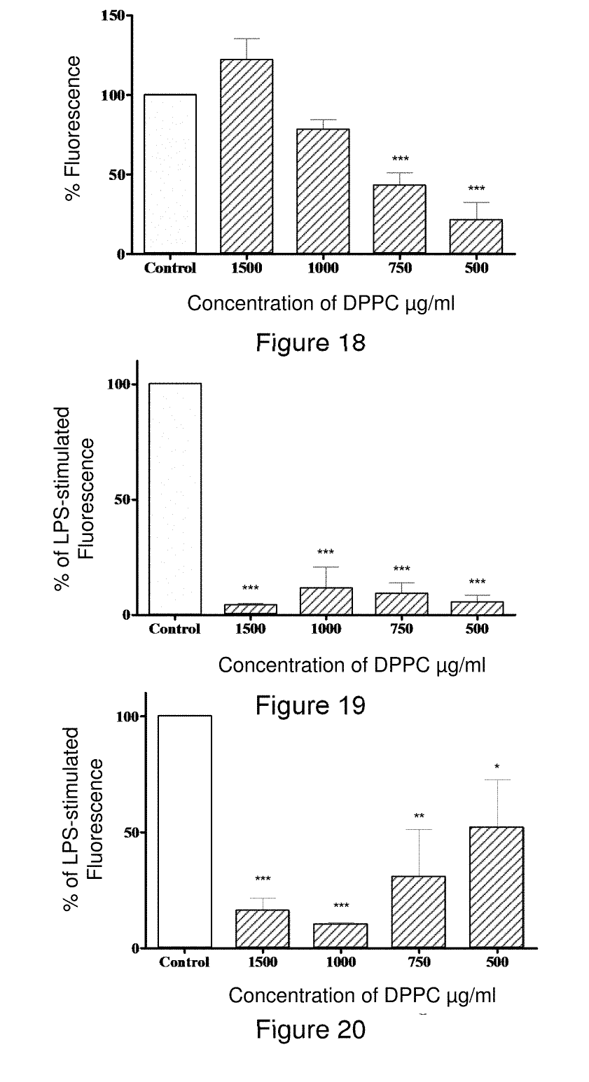

The anti-cell proliferation properties of synthetic surfactant, Synsurf®, was compared to those of animal derived surfactants in A549, which is a human lung adenocarcinoma cell line. The MTT assay was carried out to determine the dose- and time-dependent cellular metabolic activity of three pulmonary surfactants: Curosurf®, Synsurf® and Liposurf® to the A549 adenocarcinoma basal epithelial cells. FIGS. 11 to 15 show the plots of the cytotoxic response. The results show dose-dependent as well as exposure time-dependent cytotoxicity of the surfactants. Decrease in cell viability (*P—0.05) by exposure to higher phospholipid concentrations of Curosurf® and Liposurf® were witnessed at 1 h (FIG. 12). Both Synsurf® and Liposurf® displayed a similar trend in dose-dependent cytotoxicity at 4 h (FIG. 13) that was also seen in the NR8383 cell line (FIG. 8). Synsurf® exhibited the same trend for the 12 h (FIGS. 14) and 24 h (FIG. 15) exposure time. Significant decrease in cell viability was seen at phospholipid (DPPC) concentrations of 1000 μg/ml (**P≤0.01) and 1500 μg/ml (***P≤0.001) for 12 h and at phospholipid (DPPC) concentrations of 750 μg/ml (*P<0.05), 1000 μg/ml (***P≤0.001) and 1500 μg/ml (***P≤0.001) for 24 h.

Synsurf® inhibited A549 cell growth and decreased cell viability monitored from 1 h to 24 h in a dose-dependent manner. It is clear from at least FIGS. 14 and 15 that Synsurf® significantly decreases cell viability of the adenocarcinoma cell line A549 in a dose dependent and time-exposure dependent manner. Cell viability is reduced by more than 50% at a Synsurf® concentration of 1500 μg/ml after 12 h or 24 h of exposure to Synsurf®. Synsurf® also inhibited cell proliferation in dose- and time-dependent manner as measured by 3-[4,5-methylthiazol-2-yl]-2,5-diphenyl-tetrazolium bromide assay at 24 hours. The synthetic surfactant affects the proliferation of pulmonary epithelium and this effect is dependent on the dose and duration of exposure. Synsurf® can thus be used in the treatment of cell proliferation disorders such as lung cancer.

Accordingly, Synsurf®, is chemotherapeutic when exposed to the adenocarcinoma cell line A549. Synsurf® appears to be more chemotherapeutic compared to other naturally derived animal surfactants. Liposurf® decreased the adenocarcinoma cell line A549 viability by an appreciable amount (approximately 20%) after exposure of the cell line for 4 hours.

### Oxidative Burst

1. NR8383 Rat Alveolar Macrophages

Both animal derived surfactants significantly decreased basal levels of reactive oxygen intermediates at phospholipid concentration of 500-1500 μg/ml compared to the un-treated & un-stimulated AMs (FIGS. 16 and 17). Curosurf® significantly decreased (P≤0.0001) ROS production by 90.05±0.45%-94.75±1.82%, and Liposurf® decreased (P≤0.0001) ROS production by 89.1±1.20%-96.16±0.44%. No statistical differences were found among the varying concentrations nor were differences found between the separate surfactants. Synsurf® increased ROS production by 21.6±13.89% at the phospholipid concentration 1500 μg/ml (FIG. 17) but significantly decreased (P≤0.001) ROS production by 22.00±3.77%-80.14±6.30% in a dose-dependent manner at decreasing phospholipid concentration of 750-500 μg/ml.

The animal derived surfactants, Curosurf® and Liposurf®, and the synthetic surfactant (Synsurf®) significantly decreased basal levels of oxidative burst at phospholipid concentration of 500-1500 μg/ml compared to the LPS-stimulated AMs (FIGS. 19 to 21). Curosurf® significantly decreased (P≤0.0001) ROS production by 88.53±9.20%-95.92±0.81%, and Liposurf® decreased (P≤0.0001) ROS production by 48.17±20.7%-89.8±0.85% in a dose-dependent manner. No statistical differences among the varying concentrations were found for Curosurf®. However, a significant increase (P≤0.05) in ROS production was seen between phospholipid concentrations 1000 μg/ml vs 500 μg/ml for Liposurf®. On the other hand, Synsurf® decreased ROS production by 62.43±21.58%-88.37±055% at phospholipid concentration of 500-1500 μg/ml compared to the LPS-stimulated AMs (FIG. 21). No statistical differences among the varying concentrations were found thus displaying a threshold effect.

2. A549 Lung Carcinoma

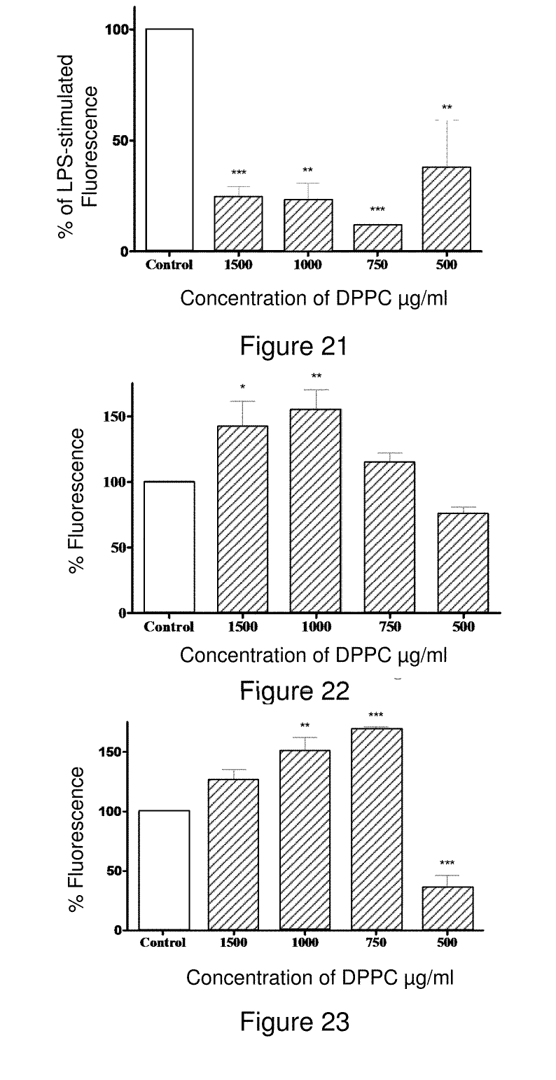

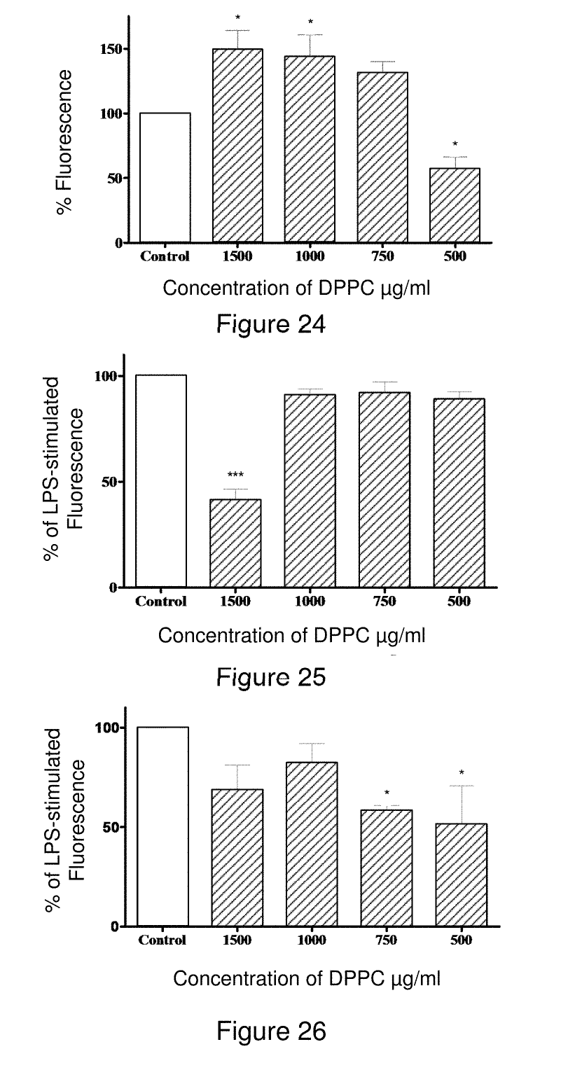

All three exogenous surfactants significantly increased basal levels of reactive oxygen intermediates at high phospholipid concentration of 1000-1500 μg/ml compared to the un-treated & un-stimulated type II epithelia cells. Curosurf® (FIG. 22) significantly increased (P≤0.05) ROS production by 42.14±19.11% at phospholipid concentration of 1500 μg/ml and 55.08±18.16% at phospholipid concentration of 1000 μg/ml (P≤0.01). No statistical differences were found among the varying concentrations nor were differences found between the separate surfactants. Liposurf® (FIG. 23) increased (P≤0.01) ROS production by 49.6±12.16% at phospholipid concentration of 1000 μg/ml and continued that increase to 69.0±2.11% at 750 μg/ml phospholipids (P≤0.001). Interestingly, at 500 μg/ml phospholipids, ROS significantly decreased by 63.99±10.20% compared to the control (P≤0.001). Synsurf® (FIG. 24) increased (P≤0.05) ROS production by 49.5±14.39% at phospholipid concentration of 1500 μg/ml and 43.8±16.84% at 1000 μg/ml phospholipids (P≤0.05) but significantly decreased (P≤0.05) ROS production by 43.51±9.76% at 500 μg/ml phospholipids compared to the control.

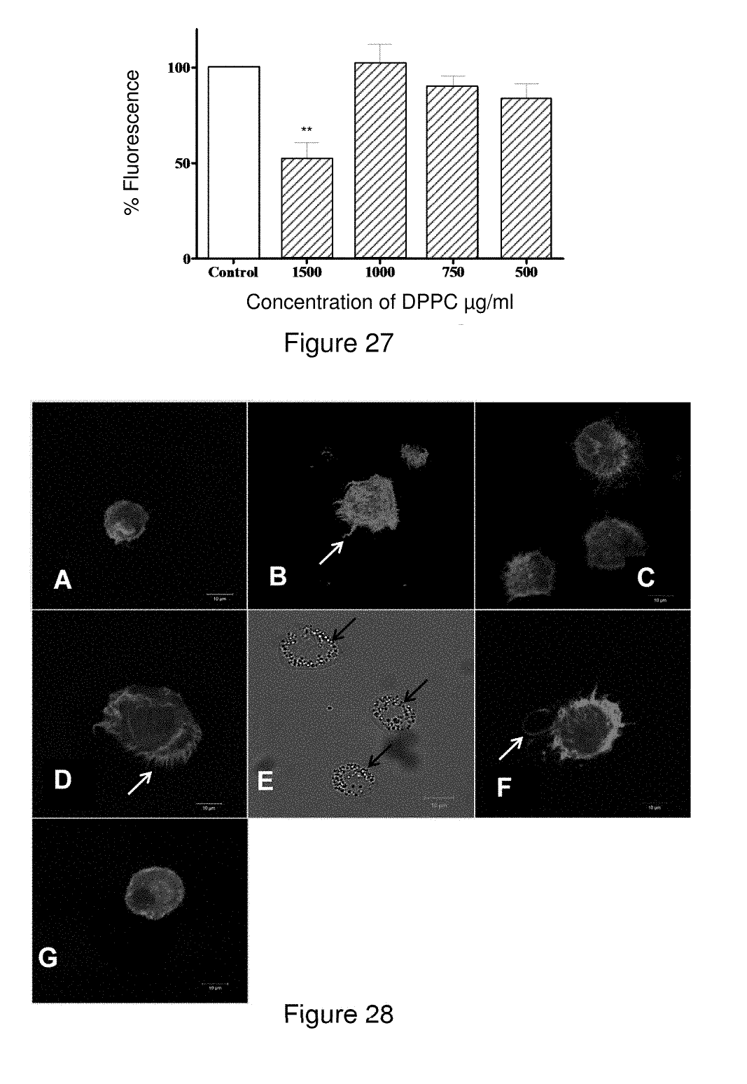

Curosurf® (FIG. 25) and Synsurf® (FIG. 27) significantly decreased basal levels of oxidative burst at phospholipid concentration 1500 μg/ml by 58.52±5.23% (P≤0.001) and 48.05±8.81% (P≤0.01) respectively compared to the LPS-stimulated type II epithelial cells. The phospholipid concentrations 1000-500 μg/ml for both Curosurf® and Synsurf® displayed no effect on LPS stimulated ROS production. On the other hand, Liposurf® decreased ROS production by 41.69±2.58% (P≤0.05) and 48.63±19.45% (P≤0.05) respectively at phospholipid concentration of 750 and 500 μg/ml compared to the LPS-stimulated type II epithelial cells (FIG. 26). No statistical differences were found among the varying concentrations.

### Correlations

To determine whether the oxidative damage from intracellular ROS production is correlated with cell viability, Pearson's nonparametric correlation was performed (Table 2 below) for the three surfactants at their respective phospholipid concentrations (500-1500 μg/ml) following the 24 h time exposure. Graphs were also plotted to correlate the observed increases in the percentages of cells containing high intracellular ROS levels and viable cell percentages. Synsurf® displayed a strong negative relationship between intracellular ROS levels and viable cell percentages for the NR8383 alveolar macrophages. A moderate positive linear relationship was found for Curosurf® in the A549 cell line whereas Synsurf® displayed a strong negative linear relationship between intracellular ROS levels and viable cell percentages.

TABLE 2
The Pearson's Correlation Coefficient between percentage
ROS production and percentage cell viability in all three
surfactants in both cell lines (unstimulated) at 24 h. r, correlation
coeffient; r², squared correlation coefficient; significant
correlation established at P ≤ 0.05.

| | [r] | [r²] | Description | P value |
|---|---|---|---|---|
| NR8383 | | | | |
| Curosurf ® | −0.091 | 0.008 | Weak negative linear | 0.909 |
| | | | relationship | |
| Liposurf ® | 0.068 | 0.005 | Weak Positive linear | 0.932 |
| | | | relationship | |
| Synsurf ® | −0.925 | 0.856 | Strong negative linear | 0.075 |
| | | | relationship | |
| A549 | | | | |
| Curosurf ® | 0.653 | 0.426 | Moderate positive linear | 0.347 |
| | | | relationship | |
| Liposurf ® | −0.102 | 0.011 | Weak negative linear | 0.898 |
| | | | relationship | |
| Synsurf ® | −0.761 | 0.579 | Strong negative linear | 0.240 |
| | | | relationship | |

### Stimulation of Actin Structure Formation and Polymerization in Alveolar Macrophage

In a qualitative visual assay in which deviations from the untreated state were screened, animal derived surfactants stimulated the formation of actin filled structures in a manner similar to that of the LPS stimulated macrophages. In this assay, the large majority of untreated macrophages showed only cortical staining for F-actin with little, or no, actin-filled cellular processes such as filopodia/lamellopodia (FIG. 28A). Treatment with 1 μg/ml LPS, alveolar macrophages were stimulated to polymerize actin with filopodia emanating from one side of the cell and actin-associated membrane ruffling on the other side around a flattened lamellopodia (FIG. 28B). On the other hand, Synsurf® treated macrophages (FIG. 28C) were absent of any actin polymerisation and displayed similar cortical staining to that of the untreated macrophages. At the same concentration, Curosurf® and Liposurf® treated macrophages responded with non-directional formation of many short actin filled filopodia. Liposurf® stimulated some long, actin-filled filopodia compared to that of Curosurf®. Furthermore, Curosurf® stimulated these non-directional actin-filled filopodia from the broad non-directional lamellopodia surrounding the cell and presents vacuole formation similar to that of autophagosomes.

### Discussion of the Results

Short-term MTTs are metabolic assays that measure the viability of a cell population relative to the control, untreated cells, but do not provide direct information about total cell numbers. Cells are treated with particulates for a predetermined period of time after which soluble yellow tetrazolium salts are added (3-(4,5-dimethylthiazol-2-yl)-2,5-diphenyltetrazolium bromide) for 2 h at 37° C. During this process, viable cells with active respiratory mitochondrial activity bio-reduce MTT into an insoluble purple formazan product via mitochondrial succinic dehydrogenases, which is subsequently solubilized by dimethyl sulfoxide (DMSO) or detergent and quantitated on a visible light spectrophotometer. Data are represented as optical density (OD)/control group (Hillegass, Shukla et al. 2010). To determine the in vitro effects of concentration and exposure-time of surfactant on the viability of cells that exist in the human pulmonary system (A549 and NR8383 cells), cell proliferation and cell viability was determined. The MTT assay of A549 and NR8383 cells revealed that when exposed to certain surfactants at higher phospholipid concentrations for 12 h and 24 h, there was a reduction in cell growth. Our findings show Synsurf® dose dependently inhibited the NR8383 and the A549 cell growth compared to the control cells. The morphological changes in the NR8383 cells did not indicate clear apoptotic bodies for Synsurf®, however, the Curosurf® group did show clear cytoplasmic vesicles (FIG. 28E) despite having decreased intracellular ROS production and comparative cell viability to the control group, thus indicating a ROS-independent pathway may be involved. After the 4 h time exposure, both Synsurf® and Liposurf® resulted in 73.71±9.05% and 78.48±6.20% viability in the NR8383 cell line respectively. However, only Synsurf® at the highest phospholipid concentration of 1500 μg/ml carried on with the same pattern of cell viability for the 12 h (65.79±4.5%) and 24 h (64.75±3.59%) time exposure. For the A549 cell line, Synsurf® prevented colony formation thus indicating a potential surfactant induced cellular modulation. This finding is linked to the observed increased presence of intracellular ROS with the increased presence of intracellular ROS. Reactive oxygen species are universal and pleiotropic signalling molecules implicated in the pathogenesis of disease states (Ray, Huang et al. 2012), whilst in physiological states, ROS is normally detected at low levels, required for normal cellular function (Stowe, Camara 2009). ROS is also well known as a major role player in the pathogeneses of a variety of lung disorders such as asthma, COPD, pulmonary fibrosis and cancer (Domej, Oettl et al. 2014).

The results reveal a decrease in cell viability percentage as the dose of Synsurf® increases. This suggests that the synthetic surfactant composition (phospholipids and polymers of poly-L-lysine electrostatically bonded with poly-L-glutamic acid) interacts with the cellular membranes due to its surface activity. This interaction may interfere with enzyme balance, the osmotic balance due to charge density and the presence of the cationic peptide complex or increase cell membrane permeability (Badawi, Ismail et al. 2015).

Alternatively or in addition, cellular uptake of surfactants may occur. Modified surfactant protein uptake, processing and metabolism may result in a low level increase of mitochondrial activity (5-10%) in cells exposed to certain surfactants compared to the control as seen in the MTT study.

Cell death can occur via the extrinsic and intrinsic pathways (Elmore 2007). The results suggest that DPPC in the surfactant compositions may influence death pathways as demonstrated by the induction of oxidative stress. The observation that DPPC leads to ROS generation in A549 cells suggests a possible switching from a ROS independent pathway causing cell cycle arrest (autophagy) to a ROS dependent pathway culminating in apoptotic cell death (Chairuangkitti, Lawanprasert et al. 2013). It has also been indicated that a two tiered mechanism of cell death, cell cycle arrest and ROS mediated mitochondrial dependant cell death is involved when considering the NR8383 cell line. Previous studies suggest that autophagy protects cells from caspase-independent death that occurs after Mitochondrial Outer Membrane Permeabilization (MOMP) in the presence of caspase inhibitors (or in this case, DPPC as a permeabilising agent). The results demonstrate that as long as cellular respiration occurs (in this case by increased glycolysis caused by elevated GAPDH), cells can use autophagy to survive MOMP and the release of cytochrome c and other apoptogenic proteins and recover to continue to grow (Colell, Ricci et al. 2007, Thorburn 2008). Thus, increased levels of autophagy can protect cells from apoptosis and this kind of caspase-independent death. It can be deduced that Synsurf® reduces cell viability and proliferation via the mitochondrial/caspase pathway due to the decrease in mitochondrial activity in the NR8383 cells. However, Curosurf® may elicit an autophagy pathway as the mitochondrial activity is preserved but rather the presence of autophagosomes (and not apoptotic bodies) indicate the degradation of organelles. The characterisation of cellular morphology is thus a key to distinguish the associations between the types of cell death. Apoptosis is usually due to caspase cleavage of cytoskeletal and other structural proteins; however, during autophagic cell death the cytoskeleton remains intact and is associated with accumulation of large numbers of autophagic vesicles (degraded organelles). It is therefore not surprising that there would be close connections between the regulatory mechanisms that control autophagy and apoptosis (Galluzzi, Maiuri et al. 2007, Thorburn 2008). Autophagy can also lead to cell death if cellular degradation remains unchecked. Further mechanistic studies are required to conclusively determine whether DPPC and/or surfactant protein or synthetic polypeptide interactions influence pathway switching. The results suggest that the onset of cytotoxicity, or rather cellular proliferation cessation, correlates with the activation of two intracellular stress-signalling pathways.

Next, proteomics revealed the changes in the cellular quantity of key proteins involved in cellular processes in the presence of a particular surfactant composition. Upregulation is evident where the cellular quantity of the protein is increased in the presence of a particular surfactant relative to a control. Glyceraldehyde 3-phosphate dehydrogenase which modulates the organisation and assembly of the cytoskeleton and is involved in transcription, RNA transport, DNA replication and apoptosis is upregulated in the presence of Synsurf®, Curosurf® and Liposurf®, relative to a control. MHC class 1 b molecules which bridge innate and acquired immunity is upregulated in the presence of Curosurf® and Liposurf® relative to a control. Whereas Synsurf® did not increase the cellular quantity of MHC class 1b molecules relative to the control. Apoptosis regulator BAX which accelerates programmed cell death is upregulated in the presence of Liposurf® and Synsurf® relative to the control. NOL1/NOP2 having RNA methyltransferase activity is upregulated in the presence of Synsurf® relative to a control, but downregulated relative to the control in the presence of Curosurf® and Liposurf®. Arginase I (M2) active in a sub-pathway of the of the urea cycle which is responsible for nitrogen metabolism is upregulated relative to the control in the presence of Synsurf® whereas it is downregulated relative to the control in the presence of Curosurf® and Liposurf®. The proteomics have thus revealed that exogenous surfactants are involved in the regulation of M1/M2 switch pathways. This means that macrophage phenotype expression can be modified or regulated when treating inflammatory conditions with the surfactants, leading the way to personalised and precision medicine.

Proteomics data were obtained and analysed in relation to LPS-stimulated AMs exposed to Synsurf®. Table 3 below lists the proteins that are only expressed in LPS-stimulated AMs exposed to Synsurf®.

TABLE 3
List of proteins expressed in the Synsurf ® exposed
LPS-stimulated AMs only.

| Identified Proteins | Accession Number |
|---|---|
| Voltage-dependent anion-selective | VDAC1_RAT |
| channel protein 1 | |
| Peroxiredoxin-1 | PRDX1_RAT |
| Synaptic vesicle membrane | VAT1_RAT |
| protein VAT-1 homolog | |
| Ab2-162 | Q7TP54_RAT |
| Thioredoxin | THIO_RAT |
| Protein S100-A10 | S10AA_RAT |
| Ferritin (Fragment) | Q5FVS1_RAT |
| Succinate dehydrogenase | SDHA_RAT |
| [ubiquinone] flavoprotein | |
| subunit, mitochondrial | |
| LDLR chaperone MESD | MESD_RAT |
| Protein Itih2 | D3ZFH5_RAT |
| Aldose reductase | ALDR_RAT |
| Complement component 1 Q | C1QBP_RAT |
| subcomponent-binding | |
| protein, mitochondrial | |
| Ras-related protein Rab-11B | RB11B_RAT |
| V-type proton ATPase subunit C 1 | VATC1_RAT |
| NOL1/NOP2/Sun domain family, | D4A3S8_RAT |
| member 2 (Predicted) | |
| Protein Serpinc1 | Q5M7T5_RAT |
| Cytochrome c oxidase subunit 4 isoform 1, | COX41_RAT |
| mitochondrial | |
| Calcineurin B homologous protein 1 | CHP1_RAT |
| Growth/differentiation factor 15 | GDF15_RAT |

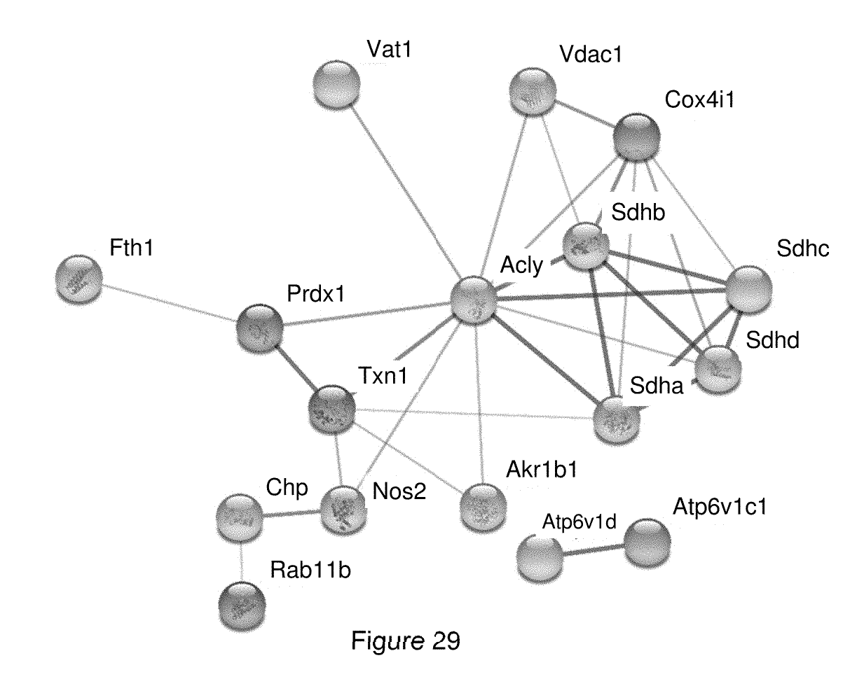

FIG. 29 shows a protein-protein interaction (PPI) network visualised by STRING for Synsurf® exposed LPS-stimulated AMs with only the associated proteins shown. The PPI network is a visualisation of the associated proteins of Table 3. The proposed statistical enrichment analysis in FIG. 29 of annotated functions for PPI was investigated further. The functional PPI enrichment (GO terms) for Synsurf® that displayed any significant enrichment value (p-value: 1.14E-06) was associated with the biological process (GO:0055114; Cox4i1, Fth1, Prdx1, Sdha, Sdhb, Txn1, Vat1) of oxidation-reduction with a FDR of 2.76E-03. This biological process is linked to the molecular function (GO:0016491; Cox4i1, Fth1, Prdx1, Sdha, Sdhb, Sdhd, Txn1, Vat1) of oxidoreductase activity (FDR 1.42E-05) and the regulation thereof.

Table 4 lists the number of up- and down-regulated proteins that are differentially expressed for the control (CTR) and Synsurf® (S) based on proteomic quantification.

TABLE 4
The up- and down-regulated proteins that are differentially expressed for the
control (CTR) and Synsurf ® (S) based on proteomic quantification (n = 3).

| | | ANOVA Test | | |
|---|---|---|---|---|
| | Accession | (P-Value) | | |
| Identified Proteins | Number | (p <= 0.05) | Quantitative Profile | |
| Heme oxygenase 1 | HMOX1_RAT | 0.0002 | CTR high*** | S low* |
| Heterogeneous nuclear | HNRPD_RAT | 0.03 | CTR high*** | S low* |
| ribonucleoprotein | | | | |
| Eukaryotic translation initiation factor 6 | IF6_RAT | 0.042 | CTR high*** | S low* |
| MHC class Ib antigen | O19445_RAT | 0.019 | CTR high*** | S low |
| Voltage-dependent anion-selective | VDAC3_RAT | 0.0066 | CTR high*** | S low* |
| channel protein 3 | | | | |
| ATP5H_RAT ATP synthase subunit d, | ATP5H_RAT | 0.022 | CTR high*** | S low* |
| mitochondrial | | | | |
| Glyceraldehyde-3-phosphate | G3P_RAT | 0.033 | CTR low* | S high*** |
| dehydrogenase | | | | |
| Cystatin-B | CYTB_RAT | 0.0051 | CTR low* | S high*** |
| MAP4_RAT Isoform 2 of Microtubule- | Q5M7W5-2 | 0.014 | CTR low* | S high*** |
| associated protein 4 | | | | |
| Calcineurin B homologous protein 1 | CHP1_RAT | 0.021 | CTR low* | S high*** |
| Pcbp2 protein | Q6AYU2_RAT | 0.026 | CTR high*** | S high*** |
| Embigin | EMB_RAT | 0.038 | CTR high*** | S high*** |
| Arginase-1 | ARGI1_RAT | 0.0018 | CTR high*** | S low*** |
| RNA-binding motif protein, X | RMXRL_RAT | 0.04 | CTR high*** | S low* |
| chromosome retrogene-like | | | | |
| Cnpy3 protein | B2RYF8_RAT | 0.037 | CTR high*** | S low* |
| Protein Tln1 | G3V852_RAT | 0.0063 | CTR low* | S low* |
| Fructose-bisphosphate aldolase A | ALDOA_RAT | 0.00054 | CTR low* | S low* |
| Plectin 6 | Q6S3A0_RAT | 0.034 | CTR low* | S low* |
| High density lipoprotein binding protein | Q3KRF2_RAT | 0.032 | CTR low* | S low* |
| (Vigilin) | | | | |
| Gamma-enolase | ENOG_RAT | <0.00010 | CTR low* | S low* |
| Chaperonin containing Tcp1, subunit | Q3MHS9_RAT | 0.013 | CTR low* | S low* |
| 6A (Zeta 1) | | | | |
| Protein Rrbp1 | F1M853_RAT | 0.013 | CTR low* | S low* |
| Protein Sptbn1 | G3V6S0_RAT | 0.027 | CTR low* | S low* |
| Perilipin | Q5U2U5_RAT | 0.00091 | CTR low* | S low* |
| Destrin | DEST_RAT | 0.033 | CTR low* | S low* |
| Spna2 protein | Q6IRK8_RAT | 0.021 | CTR low* | S low* |
| 40S ribosomal protein S12 | RS12_RAT | 0.036 | CTR low* | S low* |
| Dynamin-1-like protein | DNM1L_RAT | 0.014 | CTR low* | S low* |
| Septin-9 (Fragment) | F1LN75_RAT | 0.012 | CTR low* | S low* |
| Proliferation-associated 2G4 | Q6AYD3_RAT | 0.026 | CTR low* | S low* |
| Epithelial protein lost in neoplasm | F1LR10_RAT | 0.036 | CTR low* | S low* |
| Protein Tpd52 | F1MAB9_RAT | 0.032 | CTR low* | S low* |
| Single-stranded DNA-binding protein | G3V7K6_RAT | 0.031 | CTR low* | S low* |
| 40S ribosomal protein S10 | RS10_RAT | 0.022 | CTR low* | S low* |
| Protein Vps4b | Q4KLL7_RAT | 0.013 | CTR low* | S low* |
| ARPC5_RAT Actin-related protein 2/3 | Q4KLF8 | 0.011 | CTR low* | S low* |
| complex subunit 5 | | | | |
| Septin-2 | SEPT2_RAT | 0.04 | CTR low* | S low* |
| Protein Chd4 | E9PU01_RAT | 0.05 | CTR low* | S low* |
| Fabp4 protein | Q5XFV4_RAT | 0.012 | CTR low* | S low* |
| Isoform Crk-I of Adapter molecule crk | CRK_RAT | 0.014 | CTR low* | S low* |
| Lipoprotein lipase | LIPL_RAT | 0.036 | CTR low* | S low* |
| Crk-like protein | CRKL_RAT | 0.016 | CTR low* | S low* |
| Protein Dhx58 | D3ZD46_RAT | 0.023 | CTR low* | S low* |
| Glyceraldehyde-3-phosphate | D3ZWV2_RAT | 0.033 | CTR low* | S low* |
| dehydrogenase | | | | |
| cAMP-dependent protein kinase type II- | KAP3_RAT | 0.029 | CTR low* | S low* |
| beta regulatory subunit | | | | |
| Granulocyte-macrophage colony | Q701L2_RAT | 0.0061 | CTR low* | S low* |
| stimulating receptor alpha | | | | |
| SEPT9_RAT Isoform 2 of Septin-9 | Q9QZR6-2 | 0.018 | CTR low* | S low* |
| D3ZUC9_RAT Oxidative-stress | D3ZUC9_RAT | 0.03 | CTR low* | S low* |
| responsive 1 (Predicted) | | | | |
| ELOB_RAT Transcription elongation | P62870 | 0.011 | CTR low* | S low* |
| factor B polypeptide 2 | | | | |
| Small glutamine-rich tetratricopeptide | SGTA_RAT | 0.001 | CTR low* | S low* |
| repeat-containing protein | | | | |
| Peroxiredoxin-1 | PRDX1_RAT | 0.028 | CTR low* | S high*** |
| Protein U2af1 | Q3KR55_RAT | 0.048 | CTR low* | S high*** |
| Protein Hnrnpa0 | F1M3H8_RAT | 0.025 | CTR low* | S high*** |
| Elongation factor 1-alpha 1 | EF1A1_RAT | 0.038 | CTR high*** | S low* |
| Actin-related protein 2 | ARP2_RAT | 0.0058 | CTR high*** | S low* |
| Unconventional myosin-Ic | MYO1C_RAT | 0.043 | CTR high*** | S low* |
| HtrA serine peptidase 2 | B0BNB9_RAT | 0.011 | CTR high*** | S low* |
| Thioredoxin | THIO_RAT | 0.019 | CTR high*** | S high*** |
| Succinate dehydrogenase [ubiquinone] | SDHA_RAT | 0.035 | CTR high*** | S high*** |
| flavoprotein subunit, mitochondrial | | | | |
| LDLR chaperone MESD | MESD_RAT | 0.00029 | CTR high*** | S high*** |
| Complement component 1 Q | C1QBP_RAT | 0.0059 | CTR high*** | S high*** |
| subcomponent-binding protein, | | | | |
| mitochondrial | | | | |
| Cytochrome c oxidase subunit 4 | COX41_RAT | 0.018 | CTR high*** | S high*** |
| isoform 1, mitochondrial | | | | |
| Lymphocyte cytosolic protein 1 OS | Q5XI38_RAT | 0.05 | CTR low* | S high*** |
| V-type proton ATPase subunit B, brain | VATB2_RAT | 0.033 | CTR low* | S high*** |
| isoform | | | | |
| 40S ribosomal protein S5 | B0BN81_RAT | 0.04 | CTR low* | S high*** |
| Catenin (Cadherin associated protein), | D3ZZZ9_RAT | 0.0027 | CTR low* | S high*** |
| delta 1 (Predicted), isoform CRA_a | | | | |
| Heterogeneous nuclear | HNRPQ_RAT | 0.017 | CTR low* | S high*** |
| ribonucleoprotein Q | | | | |
| Apoptosis regulator BAX | G3V8T9_RAT | 0.046 | CTR low* | S high*** |
| Hematopoietic cell specific Lyn | Q68FX4_RAT | 0.042 | CTR low* | S low* |
| substrate 1 | | | | |
| GTP-binding nuclear protein Ran | RAN_RAT | 0.008 | CTR low* | S low* |
| Ncf4 protein | B2RZ98_RAT | 0.012 | CTR low* | S low* |
| Protein LOC100912427 | F1LPL7_RAT | 0.019 | CTR low* | S low* |
| 40S ribosomal protein S17 | RS17_RAT | 0.0013 | CTR low* | S low* |
| Protein Tom1 | Q5XI21_RAT | 0.0098 | CTR low* | S low* |
| Elongation factor 1-delta | EF1D_RAT | 0.028 | CTR low* | S low* |
| Engulfment and cell motility 1, ced-12 | D3ZY46_RAT | 0.0047 | CTR low* | S low* |
| homolog (C. elegans) (Predicted), | | | | |
| isoform CRA_a | | | | |
| LRRGT00066 | Q6TUH8_RAT | 0.0018 | CTR low* | S low* |
| Protein Snx6 | B5DEY8_RAT | 0.01 | CTR low* | S low* |
| Protein Syap1 | Q6AYB6_RAT | 0.002 | CTR low* | S low* |
| Pyrroline-5-carboxylate reductase 3 | P5CR3_RAT | 0.0032 | CTR low* | S low* |
| AP-1 complex subunit beta-1 | G3V9N8_RAT | 0.033 | CTR low* | S low* |
| Prefoldin 5 (Predicted), isoform CRA_a | B5DFN4_RAT | 0.047 | CTR low* | S low* |
| Protein-L-isoaspartate(D-aspartate) O- | PIMT_RAT | 0.0063 | CTR low* | S low* |
| methyltransferase | | | | |
| Isoform Alpha of Interleukin-18 | IL18_RAT | 0.046 | CTR low* | S low* |
| Protein Pin4 | M0RCP9_RAT | 0.019 | CTR low* | S low* |
| GrpE protein homolog 1, mitochondrial | GRPE1_RAT | 0.0023 | CTR low* | S low* |
| Protein Lsm12 | D4A8G0_RAT | 0.00053 | CTR low* | S low* |
| ATP-dependent Clp protease | M0RAD5_RAT | 0.036 | CTR low* | S low* |
| proteolytic subunit | | | | |
| MAT2B_RAT Methionine | MAT2B_RAT | 0.044 | CTR low* | S low* |
| adenosyltransferase 2 subunit beta | | | | |
| AHNAK 1 (Fragment) | Q38PF9_RAT | 0.033 | CTR low* | S low* |
| Mitochondrial import inner membrane | TIM13_RAT | 0.023 | CTR low* | S low* |
| translocase subunit Tim13 | | | | |
| Coiled-coil domain-containing protein | CCD22_RAT | 0.026 | CTR low* | S low* |
| 22 | | | | |
| Purine nucleoside phosphorylase | PNPH_RAT | 0.025 | CTR high*** | S low* |
| Lipid phosphate phosphatase-related | F1LRA5_RAT | 0.0083 | CTR high*** | S low* |
| protein type 2 | | | | |
| Catalase | CATA_RAT | 0.049 | CTR high*** | S low* |
| ADP/ATP translocase 2 | ADT2_RAT | 0.0069 | CTR high*** | S low* |
| Interferon-induced GTP-binding protein | MX1_RAT | <0.00010 | CTR high*** | S low* |
| Mx1 | | | | |
| Ras-related protein Rab-1A | E9PU16_RAT | 0.017 | CTR high*** | S low* |
| N(4)-(Beta-N-acetylglucosaminyl)-L- | ASPG_RAT | <0.00010 | CTR high*** | S low* |
| asparaginase | | | | |
| Guanylate binding protein 2 | Q5PQW8_RAT | <0.00010 | CTR high*** | S low* |
| Interferon-induced protein with | Q6AYE7_RAT | 0.00016 | CTR high*** | S low* |
| tetratricopeptide repeats 3 | | | | |
| 26S proteasome non-ATPase | PSMD1_RAT | 0.016 | CTR high*** | S low* |
| regulatory subunit 1 | | | | |
| ADP/ATP translocase 1 | Q6P9Y4_RAT | 0.0067 | CTR high*** | S low* |
| Monocyte differentiation antigen CD14 | O88955_RAT | 0.022 | CTR high*** | S low* |
| Interleukin-1 alpha | IL1A_RAT | <0.00010 | CTR high*** | S low* |
| Eukaryotic translation initiation factor | IF5A1_RAT | 0.048 | CTR low* | S low* |
| 5A-1 | | | | |
| Biliverdin reductase B (Flavin reductase | B5DF65_RAT | 0.00048 | CTR low* | S low* |
| (NADPH)) | | | | |
| Eukaryotic translation initiation factor 2, | Q6P685_RAT | 0.02 | CTR low* | S low* |
| subunit 2 (Beta) | | | | |
| Perilipin | M0RA08_RAT | 0.039 | CTR low* | S low* |
| Protein Diap1 | F1M775_RAT | 0.04 | CTR low* | S low* |
| Protein Snrpa | Q5U214_RAT | 0.013 | CTR low* | S low* |
| Glutathione S-transferase alpha-3 | GSTA3_RAT | 0.021 | CTR low* | S low* |
| Ethylmalonic encephalopathy 1 | B0BNJ4_RAT | 0.0038 | CTR low* | S low* |
| WW domain binding protein 2, isoform | G3V721_RAT | 0.019 | CTR low* | S low* |
| CRA_b | | | | |
| Syntaxin-7 | STX7_RAT | 0.047 | CTR low* | S low* |
| Cysteine and glycine-rich protein 1 | CSRP1_RAT | 0.013 | CTR low* | S low* |
| LOC684322 protein | B2RZ97_RAT | 0.002 | CTR low* | S low* |
| COP9 signalosome complex subunit 8 | CSN8_RAT | 0.042 | CTR low* | S low* |
| Clathrin light chain A | CLCA_RAT | 0.0034 | CTR low* | S low* |
| Glia maturation factor beta | GMFB_RAT | 0.0073 | CTR low* | S low* |
| Putative phospholipase B-like 2 | PLBL2_RAT | 0.00071 | CTR low* | S low* |
| Olfactory receptor | Q6ZMA1_RAT | 0.00022 | CTR low* | S low* |
| Voltage-dependent anion-selective | VDAC1_RAT | 0.00067 | CTR low* | S high*** |
| channel protein 1 | | | | |
| Synaptic vesicle membrane protein | VAT1_RAT | 0.026 | CTR low* | S high*** |
| VAT-1 homolog | | | | |
| Ab2-162 | Q7TP54_RAT | 0.0011 | CTR low* | S high*** |
| Protein S100-A10 | S10AA_RAT | 0.021 | CTR low* | S high*** |
| Ferritin (Fragment) | Q5FVS1_RAT | 0.0013 | CTR low* | S high*** |
| Protein Itih2 | D3ZFH5_RAT | 0.015 | CTR low* | S high*** |
| Aldose reductase | ALDR_RAT | 0.023 | CTR low* | S high*** |
| Ras-related protein Rab-11B | RB11B_RAT | 0.014 | CTR low* | S high*** |
| V-type proton ATPase subunit C 1 | VATC1_RAT | 0.011 | CTR low* | S high*** |
| NOL1/NOP2/Sun domain family, | D4A3S8_RAT | 0.038 | CTR low* | S high*** |
| member 2 (Predicted) | | | | |
| Protein Serpinc1 | Q5M7T5_RAT | 0.038 | CTR low* | S high*** |
| Growth/differentiation factor 15 | GDF15_RAT | <0.00010 | CTR low* | S high*** |

Key:
Down-regulated expression*,
Up-regulated expression***

The only statistical functional enrichment for Synsurf® was associated with oxidation-reduction. ROS is known for being a down-stream by-product of the inflammatory response and contributes to cell viability. It also serves in the multifaceted regulation of inflammatory processes via physiological roles in signalling. One of the key role role-players that was found to be upregulated in the Synsurf® group was Peroxiredoxin-1 (Prx1). ROS production is critical for appropriate cellular responses to prevent further oxidative damage and to maintain cell survival. However, when an excessive amount of cell damage has occurred the cell usually enters a form of cell death (autophagy or apoptosis) (Morgan, Liu 2011). Prx1 belongs to a family of anti-oxidants that protects the cell from metabolically produced ROS that trigger toxic mechanisms within the cell if the signal is exacerbated, continues, or if it occurs at the wrong time and region of the cell (Nathan, Cunningham-Bussel 2013). In this context, Prx1 appears to have an unanticipated, but yet, a very specific and important role in Synsurf® exposed LPS stimulated macrophages (Robinson, Hutchinson et al. 2010). Prx1 may also contribute to the modulation of immune responses by involving Th2-responses via the induction of alternatively activated macrophages (Knoops, Argyropoulou et al. 2016). It has also been proposed by Kim et. al. that Prx1 may also inhibit NO production by suppressing ROS/NF-κB/iNOS (NOS2) signalling pathway (Kim, Park et al. 2013). When taking the visualised PPI network for Synsurf® (FIG. 29) into consideration and the association with NOS2 via Thioredoxin 1 (Trx1), it is likely that the decrease in cytokine production in the Synsurf® exposed LPS stimulated macrophages occurs via blocking the induction of the NF-κB transcription pathway by the upregulation of Prx1.

Trx1 is another oxidative, stress-limiting protein that was found to be upregulated within the Synsurf® group. It is one of the most important cellular antioxidants with anti-inflammatory and anti-apoptotic properties that is also regulated by NF-κB (Djavaheri-Mergny, Javelaud et al. 2004) but is also, in turn, able to regulate NF-κB activity through cytokine-mediated denitrosylation (-nitrosyl (-SNO)) of the p65 subunit (Kelleher, Sha et al. 2014). Kelleher and colleagues demonstrated quantification of Trx protein expression, SNO-p65 formation and NF-κB activity in lung cell lysates and found that Trx legitimates cytokine-mediated denitrosylation of p65 and therefore its role in NF-κB activation associated with LPS-induced airway inflammation.

Trx-1 may also promote macrophage differentiation into the macrophage M2 anti-inflammatory phenotype. Thereby significantly reducing the LPS induced inflammatory M1 macrophages as indicated by the Synsurf® dose-dependent decrease cytokine expression of the M1 macrophages, TNFa and IL1β. This is supported by similar findings by El Hadri et al. and Billiet et al. where both groups demonstrated the induced downregulation of nuclear translocation of activator protein-1 and Ref-1 via Trx-1 that led to the shift in phenotype pattern of lesional macrophages to predominantly M2 over M1 and subsequently the secretion of pro-inflammatory cytokines (Billiet, Furman et al. 2005, El Hadri, Mahmood et al. 2012). It is then practical to assume that Synsurf® exposed LPS stimulated AMs upregulates Trx1 activity and therefore induces Trx-dependent denitrosylation of the NF-κB p65 subunit; consequently, downregulating the transcription of pro-inflammatory cytokines via the ERK MAP kinase pathway.

Moreover, Trx can be regarded as an adaptive response as it possibly acts as a chaperone to arginase by protecting the enzyme from inhibition via reactive oxygen and nitrogen intermediates (Nakamura, Nakamura et al. 2005). Thus maintaining arginase in a catalytically active state preserving its activity and blunting excessive NOS2 activity (McGee, Kumar et al. 2006, Lucas, Czikora et al. 2013). Taken together, all of these Redox activities makes Synsurf® potent and versatile mediator of inflammation and can possibly be proposed as a therapeutic candidate for the treatment of several pulmonary inflammatory disorders where Trx-1 may relieve the cytotoxic response.

Ferritin Heavy Chain (FHC) is the second-most well-known NF-κB target that protects from oxidative damage and Ferritin (Q5FVS1_RAT) was found to be co-upregulated in Synsurf® alongside the above mentioned. Due to its characteristic of being an iron storage protein, it cannot scavenge ROS directly but can, however, protect the cell from iron-mediated oxidative damage by preventing generation of highly reactive OH radicals via Fenton reactions (Morgan, Liu 2011). Thus preventing the generation of more highly reactive species (O2.- and . .OH) and promoting the breakdown of H₂O₂ into water by peroxidases and catalases (Torti, Torti 2002). However, in this case, Catalase (CATA_RAT) was downregulated in Synsurf®. Besides its ability to promote cell growth, there have been reports that suggest that catalase could be the target of inhibitory p50 homodimers since its promoter is bound by p50 in unstimulated cells and catalase is down-regulated when NF-κB activation occurs (Schreiber, Jenner et al. 2006, Morgan, Liu 2011). Thus, in this instance, it could be that, even though there is evidence that NF-κB activation via stimulated AM is decreased, it is still too high for catalase to be upregulated.

### Example 3—Drug Delivery with Dual Immunomodulatory Effects

The human respiratory tract has the potential to provide means for non-invasive drug delivery of molecules that cannot be efficiently, reproducibly, or rapidly delivered into the body. For this reason mixing of a pharmaceutically active agent with pulmonary surfactant may provide an attractive method of improving drug delivery through airway epithelium which serves as a natural barrier. Designing a successful system for drug delivery to the respiratory tract requires a comprehensive understanding of the disease condition, lung anatomy and physiology, physico-chemical properties of the drug alone, the polymeric matrix combined with its production process and the effects thereof. The future of pharmacotherapy for many disorders may lie in drug delivery routes other than oral administration. In particular, growing interest has been given to the lung as well as other absorptive mucosae as non-invasive administration routes for systemic delivery for therapeutic agents. Further interest has been shown in targeted drug delivery.

Site specific drug delivery aims to concentrate medication in the tissues of interest while reducing the relative concentration of the medication in the remaining tissues, improving the efficacy of the drug, minimizing systemic exposure and therefore circumventing adverse effects. The possibility of inhalable formulations through the pulmonary route allows for faster onset and the drug is delivered directly to the target organ. Moreover, repurposing of exogenous surfactant as a dual drug delivery agent is possible as surfactant dysfunction is usually evident in pulmonary disease states.

It has been found that the synthetic surfactant composition synergistically enhances drug efficacy by enhancing permeability of the drug through lipid barriers such as membranes to the site in the cell where it will be most effective. In particular it has been found that the synthetic surfactant composition enhances the efficacy of antibiotics in the treatment of bacterial infections. Pulmonary surfactant compositions differ to that of most eukaryotic membranes but it does contain approximately 10% PG which is a prominent component of the gram-positive bacterium's plasma membrane. Due to the thick lipid coat surrounding bacteria (consisting of trehalose dimycolate (TDM)), they are consequently well shielded from the immune system's response and antibiotics. This protective barrier may be removed by exposing the bacteria to certain surfactants. Due to multi-drug-resistant strains of bacteria emerging, there is a need for novel anti-mycobacterial agents. Surfactants have the potential to overcome natural resistance of Mycobacterium tuberculosis (M.tb) to antibiotics as they can allow better penetration of drugs through these barriers as they act as cellular permeabilizing agents associated with low toxicity.

The oxazolidinones represent a unique class of totally synthetic antimicrobial agents that were first discovered in the 1970s but re-investigated in 1996 resulting in Linezolid's discovery. For that reason, there are no pre-existing specific resistance genes among gram-positive bacteria to date. These agents also have a unique mechanism of action that precludes cross-resistance with currently available agents. Linezolid is the first oxazolidinone developed and approved for clinical use and is an inhibitor of bacterial ribosomal protein synthesis (mRNA synthesis). It prevents the formation of a 70S initiation complex by binding to domain V on the 50S ribosomal subunit near its interface with the 30S unit.

M.tb bacteria have the ability to invade and survive within the alveolar macrophage. Many compounds have been tested for their activity in vitro against M.tb strains. However, intracellular bacteria complicate optimal chemotherapy predictions. This is due to the fact that compounds depend on a series of pharmacokinetic and pharmacodynamic factors such as penetration, accumulation and bioavailability of the drugs inside cells for bacteria to be available to susceptibility. Therefore, it is of great significance that Linezolid is capable of entering alveolar macrophages and still display intracellular activity against M.tb bacteria.

### Experimental Section

Experiments were conducted to investigate alternative treatment option for pulmonary tuberculosis that alters the bacterial cell membrane, and therefore its protective barrier, by employing exogenous surfactants as vehicles for drug delivery. This will potentially allow for the antibiotics to access their targets within the organisms without rendering the drug inactive. The effects of various pulmonary surfactant on the antimicrobial agent Linezolid against M. tuberculosis are described below.

### Materials and Methods

All experiments with infectious material were carried out in a category-3 biosafety laboratory.

TABLE 5
Minimum Inhibitory Concentration of Linezolid

| | | |
|---|---|---|
| (S)-N-[[3-(3-fluoro-4-morpholinylpheny1)-2-oxo- | H37Rv | R51 |
| 5-oxazolidinyl]methyl] acetamide | Strain | Strain |

| | |
|---|---|
| 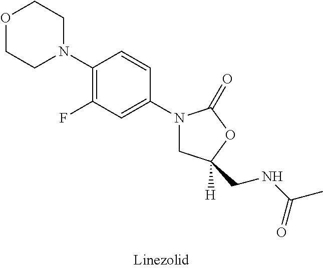 [SMILES: CC(=O)NC[C@H]1CN(c2ccc(N3CCOCC3)c(F)c2)C(=O)O1] | 1 μg/ml |

Linezolid used in this study was obtained as pure substances (Sigma-Aldrich Co., St. Louis, Mo.) and the dilution series was prepared in PBS (pH 7.4).

### Mycobacterium Species Isolates

Clinical isolates of M. tuberculosis R51 XDR strain and M. tuberculosis H37Rv reference strain, banked in the Department of Medical Biochemistry, was cultured on L-J slant cultures and used for MGIT analysis. Quality control was carried out by coexistent determination of the MICs.

### Mycobacterium Culture and Growth Inhibition

Mycobacterial growth was measured by using mycobacterial growth indicator tubes (MGIT) for drug-resistant strains from South Africa. Mycobacterial inocula were prepared from cultures of all strains grown on Lowenstein Jensen (LJ) slants. Cell suspensions were prepared in saline and the turbidity adjusted to 0.5 McFarland units. A 1:5 dilution of the bacterial suspension was prepared, and 0.5 ml of the suspension was inoculated into MGIT tubes containing test and control compounds. The MGIT 960 system (Becton Dickinson, Sparks, Md.) was used for mycobacterial growth evaluation, where M.tb growth is observed through fluorescent changes due to oxygen consumption during mycobacterial growth (Rusch-Gerdes et.al. & Becton Dickinson and Company). One-tenth milliliter of serially diluted compound was added to the MGIT tube containing 7H9 culture medium, with the final DMSO concentration not exceeding 1.2%. Incubation at 37° C. was continued in the MGIT system, and the growth units (GU) were monitored daily. For MIC₉₉ evaluations, a 1% bacterial control culture was prepared in a drug-free MGIT tube and the MIC₉₉ of the compound determined relative to the growth units of the control (GU=400). When the GU of the growth control were 400 and the GU of the drug-containing tube were more than 400, the results were defined as showing resistance, and when the GU of the drug-containing tube were less than 400, the results were considered to show susceptibility.

### Results

TABLE 6
M. tb H37Rv clinical isolate drug susceptibility testing with Linezolid
at established MIC (1 μg/ml) with various exogenous surfactants.

| | CONCENTRATION | | GROWTH UNITS | SUSCEPTIBILITY |
|---|---|---|---|---|
| COMPOUND | (μg/ml) | | (Day 8) | MIC₉₉ (μg/ml) |
| Control | — | | 400 | |
| H37Rv with PBS solvent | — | | 13816 | |
| Linezolid | 1 | μg/ml | 377 | + |
| Linezolid | 0.5 | μg/ml | 4674 | − |
| Linezolid | 0.25 | μg/ml | 12878 | − |
| Linezolid | 0.125 | μg/ml | 12030 | − |
| Linezolid + Curosurf ® | 1 | μg/ml | 0 | + (enhanced) |
| Linezolid + Curosurf ® | 0.5 | μg/ml | 3242 | − |
| Linezolid + Curosurf ® | 0.25 | μg/ml | 13276 | − |
| Linezolid + Curosurf ® | 0.125 | μg/ml | 12180 | − |
| Linezolid + Synsurf ® | 1 | μg/ml | 94 | + (enhanced) |
| Linezolid + Synsurf ® | 0.5 | μg/ml | 4773 | − |
| Linezolid + Synsurf ® | 0.25 | μg/ml | 12783 | − |
| Linezolid + Synsurf ® | 0.125 | μg/ml | 14515 | − |
| Linezolid + Liposurf ® | 1 | μg/ml | 143 | + (enhanced) |
| Linezolid + Liposurf ® | 0.5 | μg/ml | 3706 | − |
| Linezolid + Liposurf ® | 0.25 | μg/ml | 13176 | − |
| Linezolid + Liposurf ® | 0.125 | μg/ml | 11228 | − |

| | | | |
|---|---|---|---|
| Curosurf ® only | 1% | 10674 | − |
| Synsurf ® only | 1% | 17589 | − |
| Liposurf ® only | 1% | 14252 | − |

TABLE 7
M. tb X51 clinical isolate drug susceptibility testing with Linezolid
at established MIC (1 μg/ml) with various exogenous surfactants.

| | CONCENTRATION | | GROWTH UNITS | SUSCEPTIBILITY |
|---|---|---|---|---|
| COMPOUND | (μg/ml) | | (Day 8) | MIC₉₉ (μg/ml) |
| Control | — | | 400 | |
| X51 with PBS solvent | — | | 12861 | |
| Linezolid | 1 | μg/ml | 0 | + |
| Linezolid | 0.5 | μg/ml | 714 | − |
| Linezolid | 0.25 | μg/ml | 6727 | − |
| Linezolid | 0.125 | μg/ml | 9164 | − |
| Linezolid + Curosurf ® | 1 | μg/ml | 0 | + |
| Linezolid + Curosurf ® | 0.5 | μg/ml | 622 | − |
| Linezolid + Curosurf ® | 0.25 | μg/ml | 6131 | − |
| Linezolid + Curosurf ® | 0.125 | μg/ml | 10191 | − |
| Linezolid + Synsurf ® | 1 | μg/ml | 0 | + |
| Linezolid + Synsurf ® | 0.5 | μg/ml | 1255 | − |
| Linezolid + Synsurf ® | 0.25 | μg/ml | 6316 | − |
| Linezolid + Synsurf ® | 0.125 | μg/ml | 11684 | − |
| Linezolid + Liposurf ® | 1 | μg/ml | 0 | + |
| Linezolid + Liposurf ® | 0.5 | μg/ml | 301 | + (enhanced) |
| Linezolid + Liposurf ® | 0.25 | μg/ml | 5762 | − |
| Linezolid + Liposurf ® | 0.125 | μg/ml | 9021 | − |

| | | | |
|---|---|---|---|
| Curosurf ® only | 1% | 12892 | − |
| Synsurf ® only | 1% | 13420 | − |
| Liposurf ® only | 1% | 13333 | − |

The standard H37Rv strain as well as the X51 drug resistant strain was used for the susceptibility testing of M.tb to Linezolid due to its position as a third-line drug in the treatment of MDR-TB. Furthermore, Linezolid, in combination with all three exogenous surfactants, displayed no decrease in activity to both strains compared to their unformulated counterparts in vitro. The Curosurf®-Linezolid combination displayed a total zero growth compared to Linezolid alone whereas Synsurf® in combination with Linezolid displayed a 75.1% increased susceptibility compared to Linezolid alone for the H27Rv strain at the MIC₉₉ of 1.00 μg/ml. Whereas Liposurf®-Linezolid combination displayed a 62.1% enhanced action.

Linezolid displayed a 31% and 13% increase in bacterial susceptibility when in combination with Curosurf® at 0.5 μg/ml for the H37Rv strain and X51 strain respectively. Although the strains do not display official clinical susceptibility at those concentrations, the surfactant combinations do indeed demonstrate definite influences on the respective MIC's. The Linezolid -Liposurf® combination had a distinct effect on the bacterial susceptibility as the activity displayed a 20.7% increase for the H37Rv strain at 0.5 μg/ml compared to Linezolid alone, whereas for the X51 strain, it showed complete susceptibility at half of the official MIC₉₉. Furthermore, all three surfactants displayed increased antimycobacterial activity to their relevant strains when in combination with Linezolid at the MIC₉₉ that was as effective as their unformulated counterparts in terms of their in vitro efficacy. The surfactants alone displayed no antimycobacterial activity against either strain.

The surfactant compositions act as “permeabilising agents” and are thought to be involved in removing the TDM coat that acts as the first line of defence of M.tb. For that reason, increased drug permeability is allowed into the intracellular space of the bacteria rather than an increase in the activity of the drug. Taking the surfactant proteins into consideration, SP-A and SP-D may facilitate the clearance of bacteria through numerous mechanisms. SP-A and SP-D may also act as opsonins to enhance bacterial phagocytic removal in vivo. Additionally, these collectins appear to exhibit anti-microbial effects on bacteria by potentially increasing the permeability of their membranes.

Both SP-A and SP-D bind to LPS, but each interacts with LPS via different mechanisms: SP-D binds to LPS through the core oligosaccharides, whereas SP-A binds to the lipid A domain. SP-D binds Gram-positive bacteria though peptidoglycan and lipoteichoic acid. SP-A is unable to interact with these parts but instead utilizes the extracellular adhesin protein, Eap, on Gram-positive S. aureus for opsonisation. These collectins promote phagocytosis indirectly, by stimulating the activity of alveolar macrophages. S. aureus is opsonized by SP-A, and this allows for the interaction with the recognised SP-A receptor 210 (SP-R210) on alveolar macrophages for phagocytosis. SP-R210 is also involved in the killing of Mycobacterium bovis (bacillus Calmette-Guerin) that has been opsonized by SP-A. Furthermore, SP-A could mediate how M tuberculosis attaches to AM in a [Ca2+]-dependent manner.

Administration of Linezolid combined with an exogenous surfactant leads to site-specific drug administration. This demonstrates how the reach of anti-bacterial agents can be improved, allowing for lower dosages and the subsequent prevention of undesired adverse-effects due to systemic toxicity. The use of Linezolid in anti-TB therapy has become popular due to raising concern that some strains are resistant to current chemotherapy. The use of Linezolid with exogenous surfactants as dual drug delivery agents results in a synergistic effect. It is important to consider Linezolid suppresses toxin production and inhibits the expression of virulent factors. Linezolid modulates the immune response by decreasing IL-1β, TNF-α and MIP2 (macrophage inflammatory protein 2) levels. MIP-2 induces neutrophil activation, chemotaxis, exocytosis, and respiratory burst. The results demonstrate the dual immunomodulatory effects of both of the active components or “agents”, namely Linezolid and the surfactant compositions.

The results demonstrate that the three different classes of surfactant (animal derived bovine vs porcine, and synthetic) increase the efficacy of the oxazolidinone, Linezolid against M.tb. The surfactants display an impact on the antibiotic activity regardless of their concentrations.

Generally, it is important to take the antibiotic target into consideration when wanting to employ a surfactant-associated therapy in which the surfactant acts as a delivery agent. Firstly, membrane targets are disordered whereas nuclear/intracellular targets are not. With regard to the use of antibiotics as therapeutic agents, the characteristics of different infection sites must be considered to determine antimicrobial susceptibility as these sites may also affect the pharmacodynamics of the antibiotics in question.

### Example 4—Inhalable Formulations of the Surfactant Composition for Drug Delivery

The lung is an attractive target for drug delivery due to the fact that non-invasive administration can be employed; it avoids first-pass metabolism; it results in a more rapid onset of therapeutic action; direct delivery to the site of treatment of respiratory diseases is achieved. In addition, the lungs provide a huge surface area for local drug action and systemic absorption of drugs.

While intravenous or oral administration results in high systemic drug concentrations for a short period of time, typically, a relatively low amount actually reaches the lung. A low lung-to-plasma ratio can potentially lead to treatment failure and may increase systemic side effects. Focused pulmonary delivery of drugs exposes lung tissue to drug concentration levels significantly higher compared to other routes of administration by reducing systemic toxicity.

Preparations for inhalation are liquid or solid preparations intended for administration as vapours or aerosols to the lung in order to obtain a local or systemic effect. They are designed to contain one or more active substances dissolved in a suitable vehicle. However, depending on the type of preparations, they may contain propellants, co-solvents, diluents, antimicrobial preservatives, solubilising and stabilising agents etc. and can be supplied in multi-dose or single-dose. These excipients should not affect the functions of the respiratory tract mucosa adversely. When supplied in pressurised containers, they comply with the requirements of the monograph on Pressurised pharmaceutical preparations (0523).

Preparations intended to be administered as aerosols are administered by various devices such as a nebulizer, pressurized metered-dose inhaler, and dry-powder inhalers. For metered-dose preparations the label usually indicates the delivered dose (except for preparations for which the dose has been established as a metered-dose or as a pre-dispensed dose), the number of deliveries from the inhaler to provide the minimum recommended dose, and the number of deliveries per inhaler. The label states, where applicable, the name of any added antimicrobial preservative.

An aerosol is defined herein as a relatively stable colloidal suspension of solid or liquid particles in a gas (usually air). There are three major categories of devices currently in clinical use to generate the aerosol particles: nebulizers, pressurized-metered dose inhalers (pMDI), and dry powder inhalers (DPI). The use of inhaled therapeutic aerosols is a common and established method for the treatment of lung diseases such as asthma and irreversible chronic obstructive lung disease.

Drugs administered directly to the lungs in patients with pulmonary diseases may accumulate in central rather than peripheral airways. This may be due to the physiologic properties of fluid dispersion in respiratory pathways and the alterations specific to inflammatory alterations of the airways such as bronchial hyper secretion, bronchoconstriction, and bronchial edema.

Surfactant compositions can be used as delivery agents for the delivery of therapeutic agents into the lungs. A less invasive method of administering such combined agents would be via inhalation by the generation of aerosols. Using such methods clinically relevant dosages of therapeutic drugs can be delivered to a specific target.

The efficacy of the inhaled aerosol depends upon a few factors regarding the particles that comprise the aerosol. The aerosol must be able to reach the desired site of action in the respiratory tract (i.e. pulmonary region). Effective, therapeutic concentrations of the aerosol must be delivered within a small number of breaths for a devise or formulation to be practical. The aerosol particles must be capable of releasing the drug particle at the site of action, before clearance mechanisms carry the compound away from the deposition site.

The deposition of therapeutic aerosols occurs by inertial impaction within the oropharynx and large conducting airways whereas the deposition in the small conducting airways and alveoli is due to gravitational sedimentation. However, both are determined by mode of inhalation, particle or droplet size, and the degree of airway obstruction.

There are three essential variables that need to be taken into consideration when considering inhalation therapy: (i) Precision which refers to the targeting of the drug to specific regions within the lung, preventing drug deposition and loss in the upper respiratory tract; (ii) uniform drug deposition in the lung which allows the drug dosing to be reliable even under varied conditions such as various ages and disease forms; and (iii) consistency which is achieved when the uniformity of drug deposition is existent throughout the device's life span.

The lung is a complex organ that is regulated by multiple factors such as physicochemical properties of the drug delivery system and the structure of the epithelia. Therefore, improving the drug deposition efficiency may not always result in enhanced therapeutic effects.

When an inhaled drug is deposited within the lung, its effectiveness depends on the landing site as well as the physicochemical properties of the active compound that determines its dissolution ability and resultant absorption and metabolism. Mucociliary clearance of drug particles at the upper respiratory tract takes place before dissolution and absorption at a rate of 1-2% per minute within the normal lung. This leads to a possible half-life of 1-2 hours. Macrophages are responsible for clearing drug particles that are deposited at the lower airways. This rate is however much slower than that of mucocilliary clearance. It then stands to reason that insoluble particles are able to reside within the alveoli for days before being completely cleared via phagocytosis.

The dissolution of particles is formally defined as a process by which the solid substances become solutes, or dissolved substances whereby the separate molecules are released in singular form depending on the interaction between the particle and the solvent. Dissolution testing has been employed for the evaluation of solid as well as semi-solid dosage forms and has become an official test in Pharmacopoeias as it allows for the reliable prediction and investigation of in vitro dissolution behaviour regarding in vivo pharmaceutical dosages.

After drugs are deposited via aerosols, they must dissolve within the lung lining fluid before cellular uptake and/or absorption can take place. The dissolution of drugs can be altered due to aqueous solubility being influenced by crystal forms, formulations and the aerosol generation mechanism. Furthermore, the limiting volume of the fluid lining may result in incomplete dissolution of the deposited drug aerosols. One can therefore deduce that the kinetics of aerosol drug dissolution on the lung surface can differ from that of mass drug materials in aqueous media and the in situ rates are formative for lung biopharmaceutics and pharmacodynamics.

The airway epithelium, made up of the lumen and submucosal tissue, of the conducting airways, is the designated site of action for many drugs. However, its physical features, metabolic activity and drug efflux systems constitute a barrier against drug absorption and are therefore a vital component for the study of drug transport and potential site for drug toxicity. Thus, an ideal model for investigating the cellular response airway epithelial cells have to respiratory pathogens or deposited particles ought to have apical and basolateral surfaces.

The differentiated normal human bronchial epithelial cells (NHBE) is an example of such a model. It however, requires excised lung tissue followed by isolation and culturing of the epithelial cells. The cells then require time to generate an air-liquid interface culture. The human bronchial adenocarcinoma, Calu-3 cell line, can be reproduced by a simple method. It has similarity to in vivo physiology to form well differentiated, polarized, electrically resistant cell layers. It has become the alternative investigatory model for the in vitro study of proximal airway respiratory exposure to better understand determinants that influences pulmonary drug dissolution of pharmacological active compounds, absorption, metabolism and efficacy.

Although the preferred method to evaluate drug deposition is in vivo analyses, in vitro cell models are appropriate systems to study the distribution, absorption and metabolism, localization and retention time of drugs and carrier systems on airway epithelium at a cellular level.

Experimental section
Materials and methods

### Calu-3 Cell Culture

The Calu-3 cell line (ATCC® HTB-55™) was first cultured as a polarized liquid-covered culture in 75 cm² flasks and maintained in a humidified, 5% CO₂-95% atmospheric air incubator at 37° C. before subcultured in 12 cm diameter Transwell© inserts (0.33 cm² polyester, 0.4 μm pore size) from Costar. Culture medium comprised of Dulbecco's modified Eagle's medium (DMEM) supplemented with 10% fetal calf serum, 1% nonessential amino acid solution (×100), 1% L-glutamine solution (200 mM), and 1% Penicilin-Streptomycin (PENSTREP).

Cells cultured on Transwell© cell culture supports using AIC (Air-Interface Culture) were seeded at a density of 5×10⁵ cells/cm² and were introduced into the apical surface of the Transwell cell culture support in 0.2 mL medium with 2 mL medium added to the basolateral chamber. The cells were incubated at 37° C., 5% CO₂ for 24 hours. After this time, medium was aspirated from the apical chamber. The cell layers were evaluated through light microscopy with a Nikon TMS Inverted Phase Contrast Microscope (Nikon, Japan) and transepithelial electrical resistance (TEER) measured using an EVOM2 chopstick electrode and EVOM2 Epithelial Voltohmeter (world Precision Instruments, USA). Pre-warmed medium (0.2 mL, 37° C.) was added to the apical chamber before returning them to the incubator to equilibrate for a further 30 minutes, and then measuring the electrical resistance. TEER was calculated by subtracting the resistance of a cell-free culture insert with correction for the surface area of the Transwell cell culture support. Finally, alcian blue staining was employed for the detection of glycoproteins.

Synsurf® pMDIs

Canisters were filled with at a 1:1 ratio of drug with Synsurf® preparations and Linezolid (Sigma Aldrich). Hydrofluoroalkane (HFA) propellant (Mexichem-Fluor, Runcorn) was added and sealed with a Pamasol Manual Crimper and Filler (DH Industries, Essex, UK).

### Scanning Electron Microscopy

The morphology of the various powder formulations was investigated using a scanning electron Microscopy (SEM) to further evaluate the drug deposition of the pMDI using the JOEL SEM6480LV system (University of Bath). Samples were gold-coated prior to imaging (Edwards Sputter Coater, Crawley, UK).

### nGI: In Vitro Aerosol Deposition Studies and Dissolution Analysis

The Next Generation Impactor™ (Copley Scientific), a high-performance cascade impactor for classifying aerosol particle into size fractions for testing metered-dose inhalers. The loaded device was connected to the throat of the nGl via a moulded mouthpiece. Testing was performed at 60 L·min⁻¹ flow rate with a 5 second exposure after each “puff”. Dissolution assay was performed upon each actuation (n=2). HBSS buffer, Sigma Aldrich (St. Louis, Mo., USA), containing a 0.025% TWEEN was used for the dissolution assay in the basolateral snapwell chamber. The impaction plates of the nGl were modified to accommodate up to eight Snapwells, 4 stages (only 3 stages were used in this study in duplicates). The aerodynamic particle size deposition and distribution of the Synsurf®-Linzolid microparticles across sub-bronchial epithelial Calu-3 cells were also studied.

### Sample Preparation

Samples from dissolution assays were vortexed for 30 seconds, centrifuged on a Hermle Z160M centrifuge (LASEC, SA) for 10 minutes at 3000 rpm and the supernatant (±1 ml) was transferred to an HPLC vial.

### Reagents and Standards

HPLC grade acetonitrile (Romil, UK), MilliQ water (Millipore, Milford, Mass., USA) and ammonium acetate (Analytical, Merck, Darmstadt, Germany) were used in the mobile phase. The rest of the analyte standards and Linezolid were supplied by SigmaAldrich (St. Louis, Mo., USA). Standards were made up volumetrically and appropriately diluted in 50% acetonitrile. The rest of the standards were made up to 0.005 mg/L, 0.05 mg/L, 0.5 mg/L, 2.5 mg/L, 5 mg/L, 25 mg/L, 50 mg/L and 250 mg/L to fall within the expected concentration range that it may occur in samples which had been diluted ten times.

### Liquid Chromatography-Mass Spectrometry (LC-MS) Methodology

A Waters Synapt G2 quadrupole time-of-flight mass spectrometer (Waters Corporation, Milford, Mass., USA), fitted with a Waters Acquity UPLC and photo diode array detector (PDA) was used for LC-MS analyses. Separation was achieved on a Waters BEH Amide UPLC column (2.1×100 mm, 1.7 μm) at a temperature of 35° C. Solvent A consisted of 10 mM Ammonium acetate in water, solvent B consisted of 10mM Ammonium acetate in 95% acetonitrile. The gradient consisted of a flow rate 0.25 ml/min, starting with 95% B to 40% B over 9 minutes, applying gradient curve 7, followed by re-equilibration to initial conditions over 5 minutes. Electrospray ionization was applied in the negative mode, using a capillary voltage of 2.5 kV, a cone voltage of 15 V, desolvation temperature of 250° C. and desolvation gas (N2) flow of 650 L/h. The rest of the MS settings were optimized for best sensitivity. Data were acquired in MSE mode, consisting of a scan using an low collision energy and a scan using a collision energy ramp from 25-60 V, which has the added advantage of acquiring low energy molecular ion data as well as fragmentation data for all analytes all the time. Data was scanned using a scan rate of 0.2 seconds over the range m/z 100-1000. Leucine enkaphalin was used as lock mass for accurate mass determination on the fly using a lock mass flow rate of 0.002 ml/min, acquiring lock mass data every 20 seconds. Sodium formate was used to calibrate the instrument. The PDA detector was set to scan over the range: 220-450 nm.

### Drug Permeability

The transport of compounds across Calu-3 cells is typically expressed in terms of the apparent permeability coefficient (Pₐₚₚ) measured in the absorptive apical to basolateral (Ap-BI) direction and is calculated using Equation 1:

$${P}_{app} = \frac{dQ}{dt} \times \frac{1}{A \times {C}_{0}} ,$$

where dQ/dt is the linear transport rate of the compound, A is the surface area of the cell layer and C₀ is the initial compound concentration in the donor chamber.

### Results

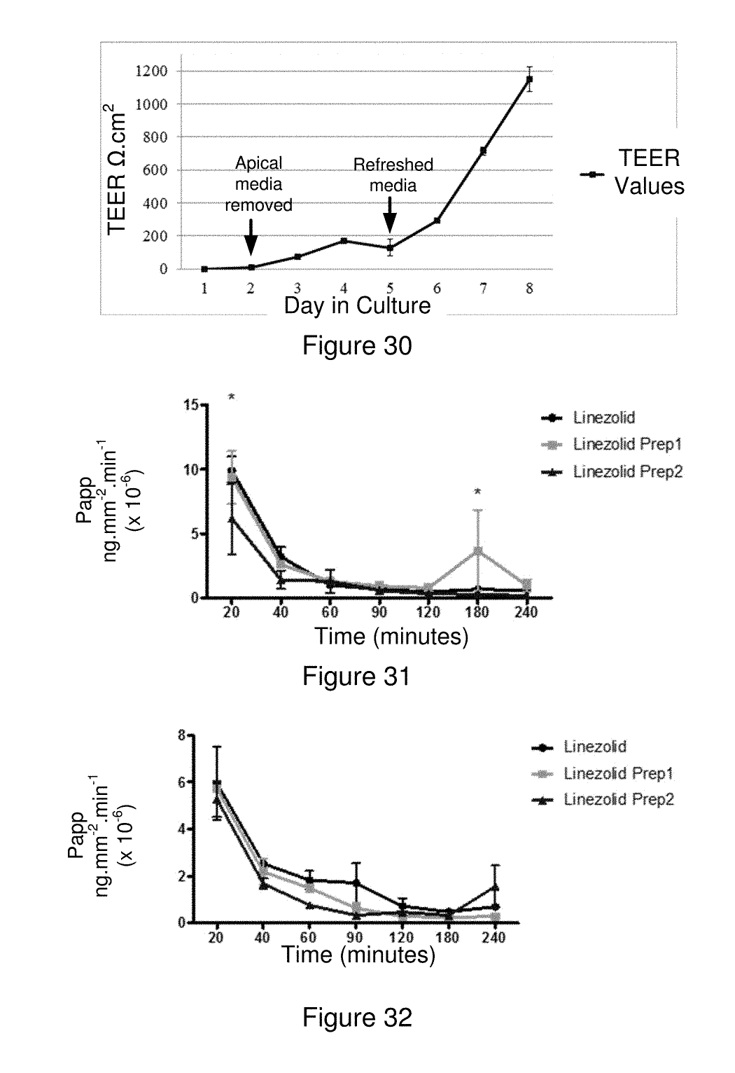

Cells grown at air-liquid interface for 6-9 days produced columnar epithelium with apical protrusions appearing to be cilia-like structures. Enhanced mucogenesis was also witnessed. The ‘tightness’ of the cell layer is determined by the tight junctions between cells, and can be assessed by measurement of TEER or the permeability of compounds transported across the cell layers exclusively by the paracellular route. Cells were deemed to have a confluent monolayer once the TEER reached 700-1000 Ω/cm². The deepening of the Alcian Blue stain indicated the increased concentration of membrane proteins from Day 1-14 in the ALI. The Trans-epithelial electrical resistance in Calu-3 cells at ALI is shown in FIG. 30.

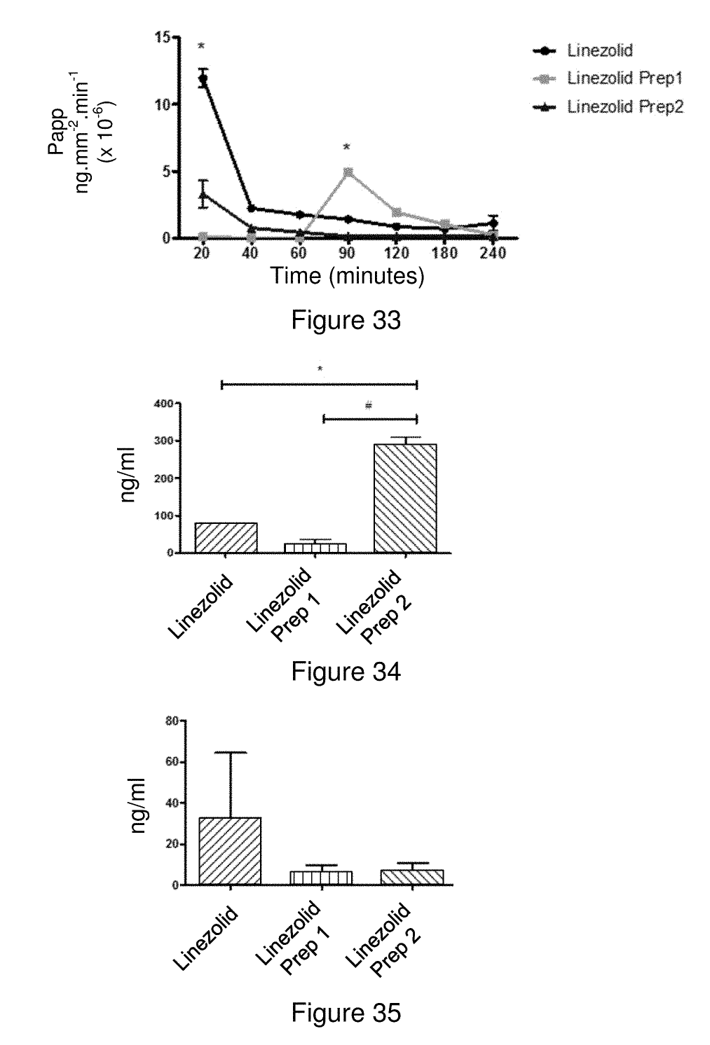

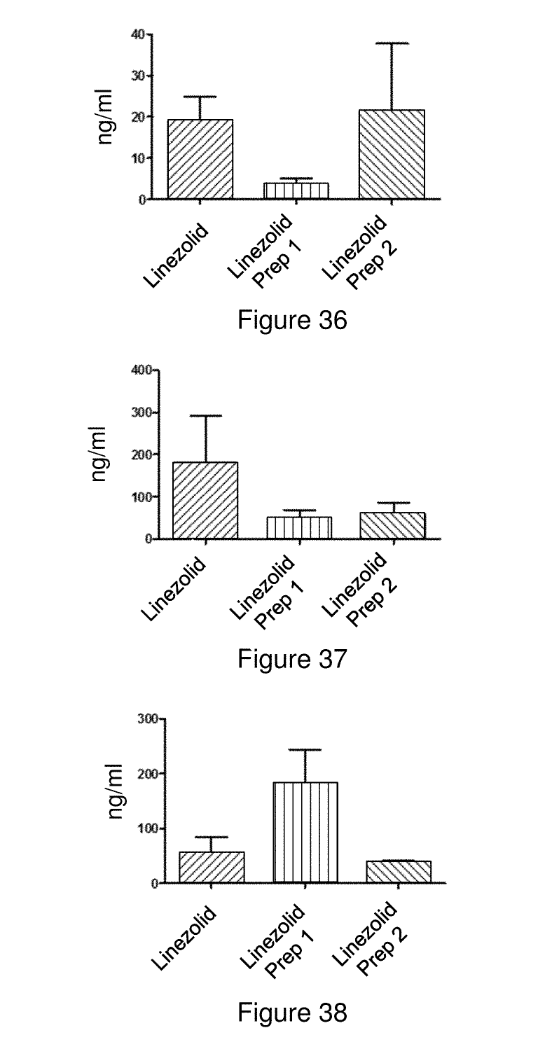

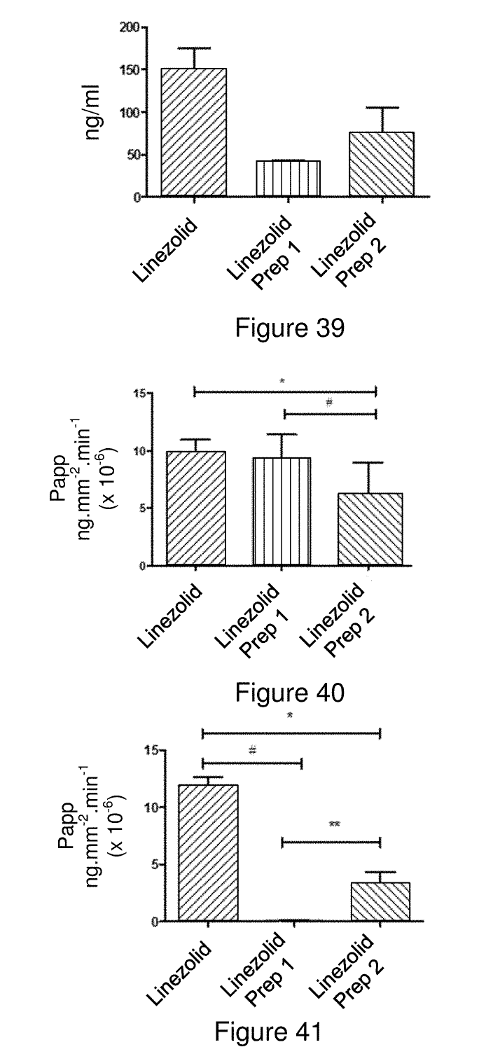

Data for each stage was collected over 240 minute periods and is plotted according to their apical to basal Pₐₚₚ values. The overall permeability coefficients (Pₐₚₚ) values measured of Linezolid, Linezolid Prep 1 and Linezolid Prep 2 across the Calu-3 transwell in Stage 2, 3 and 4 are shown in FIGS. 31 to 33 respectively. The remaining concentration values measured for Linezolid, Linezolid Prep 1 and Linezolid Prep 2 on the Calu-3 cells in Stage 2, 3 and 4 are shown in FIGS. 34 to 36 respectively. The remaining concentration values measured for Linezolid, Linezolid Prep 1 and Linezolid Prep 2 within the Calu-3 cell lysates in Stage 2, 3 and 4 are shown in FIGS. 37 to 39 respectively. The permeability coefficients (Pₐₚₚ) values measured of Linezolid, Linezolid Prep 1 and Linezolid Prep 2 across the Calu-3 transwell in Stage 2 at 20 minutes is shown in FIG. 40. FIG. 41 shows the permeability coefficients (Pₐₚₚ) values measured of Linezolid, Linezolid Prep 1 and Linezolid Prep 2 across the Calu-3 transwell in Stage 4 at 20 minutes.

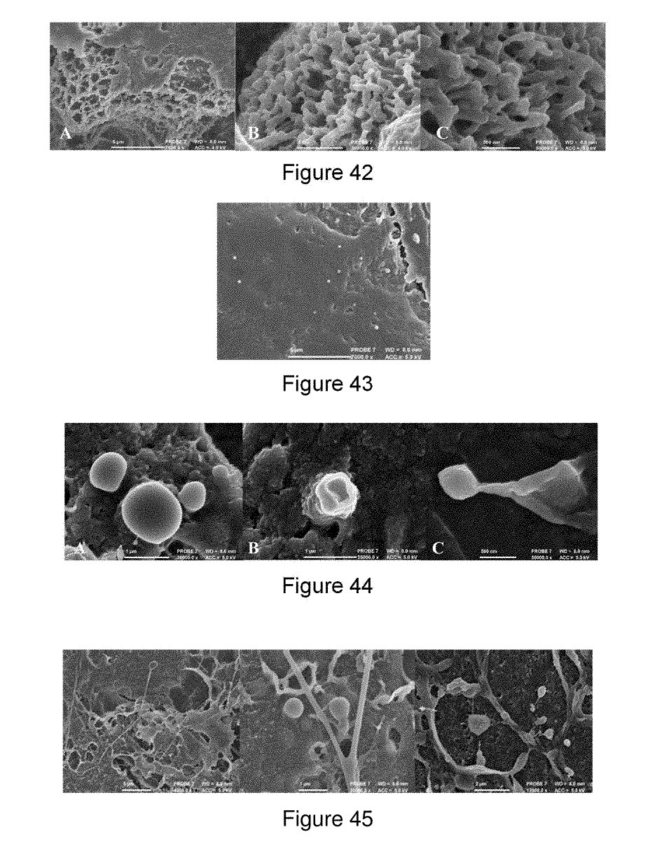

FIG. 42 shows SEM images of Calu-3 epithelial layers grown at ALI where cilia on the surface is visible as well as a mucosal layer. FIG. 43 is a SEM image showing (A) Linezolid particles deposited on top of the cells for Stage 2; and (B) examples of tight junction belt fractures after freeze-drying for SEM. FIG. 44 shows SEM images visualising the deposition of Synsurf® and Linezolid on the Calu-3 epithelial layers grown at ALI immediately post pMDI-fire. Whilst FIG. 45 shows SEM images visualising the deposition of Synsurf® on the Calu-3 epithelial layers grown at ALI. Unique spreading properties over the mucosal layers are visible 60 seconds post pMDI-fire for (A & B) Linezolid+Prep 1 and (C) Linezolid+Prep 2.

These results demonstrate that the surfactant composition, Synsurf®, can be used as a delivery agent for delivering a drug, in this case Linezolid onto epithelial layers of cells by means of a pDMI when it is suitably formulated into an inhalable formulation.

A synthetic pulmonary surfactant as described herein can be manufactured more easily, consistently and inexpensively compared to the exogenous surfactants that include animal-derived proteins or other animal-derived components. Accordingly, it will be understood by those skilled in the art that the synthetic pulmonary surfactant's mechanisms of action and potential efficacy in treating the relevant medical conditions were elucidated and evaluated by comparing the synthetic pulmonary surfactant's activity with those of the animal-derived surfactants. Any finding where the synthetic pulmonary surfactant provides comparable or better inflammatory, anti-cell proliferation, cell permeability enhancing or drug efficacy enhancing effects, for example, is an additional advantage of the synthetic surfactant over commercially available the animal-derived exogenous surfactants.

Throughout the specification and claims unless the contents requires otherwise the word ‘comprise’ or variations such as ‘comprises’ or ‘comprising’ will be understood to imply the inclusion of a stated integer or group of integers but not the exclusion of any other integer or group of integers.

### REFERENCES

BADAWI, A. M., ISMAIL, D. A., AHMED, S., MOHAMAD, A., DARDIR, M., MOHAMED, D. E., IBRAHEM, A., MANSOUR, N. A. and ASHMAWY, A., 2015. Role of Surfactants in Regulation of Cancer Growth. In: V. GANDHI, K. MEHTA, R. GROVER, S. PATHAK and B. B. AGGARWAL, eds, Multi-Targeted Approach to Treatment of Cancer. Cham: Springer International Publishing, pp. 137-149.

CHAIRUANGKITTI, P., LAWANPRASERT, S., ROYTRAKUL, S., AUEVIRIYAVIT, S., PHUMMIRATCH, D., KULTHONG, K., CHANVORACHOTE, P. and MANIRATANACHOTE, R., 2013. Silver nanoparticles induce toxicity in A549 cells via ROS-dependent and ROS-independent pathways. Toxicology in vitro, 27(1), pp. 330-338.

CHEN, N. Y., LAI, H. H., HSU, T. H., LIN, F. Y., CHEN, J. Z. and LO, H. C., 2008. Induction of apoptosis in human lung carcinoma A549 epithelial cells with an ethanol extract of Tremella mesenterica. Bioscience, biotechnology, and biochemistry, 72(5), pp. 1283-1289.

COLELL, A., RICCI, J. E., TAIT, S., MILASTA, S., MAURER, U., BOUCHIER-HAYES, L., FITZGERALD, P., GUIO-CARRION, A., WATERHOUSE, N. J., LI, C. W. and MARI, B., 2007. GAPDH and autophagy preserve survival after apoptotic cytochrome c release in the absence of caspase activation. Cell, 129(5), pp. 983-997.

DOMEJ, W., OETTL, K. and RENNER, W., 2014. Oxidative stress and free radicals in COPD-implications and relevance for treatment. International journal of chronic obstructive pulmonary disease, 9, pp. 1207.

ELMORE, S., 2007. Apoptosis: a review of programmed cell death. Toxicologic pathology, 35(4), pp. 495-516.

FAN, T. J., HAN, L. H., CONG, R. S. and LIANG, J., 2005. Caspase family proteases and apoptosis. Acta Biochimica et Biophysica Sinica, 37, pp. 719-727.

GALLUZZI, L., MAIURI, M. C., VITALE, I., ZISCHKA, H., CASTEDO, M., ZITVOGEL, L. and KROEMER, G., 2007. Cell death modalities: classification and pathophysiological implications. Cell death and differentiation, 14(7), pp. 1237-1243.

HERZOG, E., BYRNE, H. J., DAVOREN, M., CASEY, A., DUSCHL, A. and OOSTINGH, G. J., 2009. Dispersion medium modulates oxidative stress response of human lung epithelial cells upon exposure to carbon nanomaterial samples. Toxicology and Applied Pharmacology, 236(3), pp. 276-281.

HILLEGASS, J. M., SHUKLA, A., LATHROP, S. A., MACPHERSON, M. B., FUKAGAWA, N. K. and MOSSMAN, B. T., 2010. Assessing nanotoxicity in cells in vitro. Wiley Interdisciplinary Reviews: Nanomedicine and Nanobiotechnology, 2(3), pp. 219-231.

RAY, P. D., HUANG, B. W. and TSUJI, Y., 2012. Reactive oxygen species (ROS) homeostasis and redox regulation in cellular signaling. Cellular signalling, 24(5), pp. 981-990.

STOWE, D. F. and CAMARA, A. K., 2009. Mitochondrial reactive oxygen species production in excitable cells: modulators of mitochondrial and cell function. Antioxidants & redox signaling, 11(6), pp. 1373-1414.

THORBURN, A., 2008. Apoptosis and autophagy: regulatory connections between two supposedly different processes. Apoptosis, 13(1), pp. 1-9.

WALLACE, W., KEANE, M., MURRAY, D., CHISHOLM, W., MAYNARD, A. and ONG, T. M., 2007. Phospholipid lung surfactant and nanoparticle surface toxicity: lessons from diesel soots and silicate dusts. Nanotechnology and Occupational Health, pp. 23-38.

BILLIET, L., FURMAN, C., LARIGAUDERIE, G., COPIN, C., BRAND, K., FRUCHART, J. C. and ROUIS, M., 2005. Extracellular human thioredoxin-1 inhibits lipopolysaccharide-induced interleukin-1beta expression in human monocyte-derived macrophages. J Biol Chem., 280(48), pp. 40310-40318.

DJAVAHERI-MERGNY, M., JAVELAUD, D., WIETZERBIN, J. and BESANçON, F., 2004. NF-κB activation prevents apoptotic oxidative stress via an increase of both thioredoxin and MnSOD levels in TNFa-treated Ewing sarcoma cells. FEBS letters, 578(1-2), pp. 111-115.

EL HADRI, K., MAHMOOD, D. F. D., COUCHIE, D., JGUIRIM-SOUISSI, I., GENZE, F., DIDEROT, V., SYROVETS, T., LUNOV, O., SIMMET, T. and ROUIS, M., 2012. Thioredoxin-1 promotes anti-inflammatory macrophages of the M2 phenotype and antagonizes atherosclerosis. Arteriosclerosis, thrombosis, and vascular biology, 32(6), pp. 1445-1452.

KELLEHER, Z. T., SHA, Y., FOSTER, M. W., FOSTER, W. M., FORRESTER, M. T. and MARSHALL, H. E., 2014. Thioredoxin-mediated denitrosylation regulates cytokine-induced nuclear factor κB (NF-κB) activation. Journal of Biological Chemistry, 289(5), pp. 3066-3072.

KIM, S. U., PARK, Y. H., MIN, J. S., SUN, H. N., HAN, Y. H., HUA, J. M., LEE, T. H., LEE, S. R., CHANG, K. T., KANG, S. W. and KIM, J. M., 2013. Peroxiredoxin I is a ROS/p38 MAPK-dependent inducible antioxidant that regulates NF-κB-mediated iNOS induction and microglial activation. Journal of neuroimmunology, 259(1), pp. 26-36.

KNOOPS, B., ARGYROPOULOU, V., BECKER, S., FERTÉ, L. and KUZNETSOVA, O., 2016. Multiple roles of peroxiredoxins in inflammation. Molecules and cells, 39(1), pp. 60-64.

LUCAS, R., CZIKORA, I., SRIDHAR, S., ZEMSKOV, E. A., OSEGHALE, A., CIRCO, S., CEDERBAUM, S. D., CHAKRABORTY, T., FULTON, D. J., CALDWELL, R. W. and ROMERO, M. J., 2013. Arginase 1: an unexpected mediator of pulmonary capillary barrier dysfunction in models of acute lung injury. Frontiers in immunology, 4(228), pp. 1-7.

MCGEE, D. J., KUMAR, S., VIATOR, R. J., BOLLAND, J. R., RUIZ, J., SPADAFORA, D., TESTERMAN, T. L., KELLY, D. J., PANNELL, L. K. and WINDLE, H. J., 2006. Helicobacter pylori thioredoxin is an arginase chaperone and guardian against oxidative and nitrosative stresses. Journal of Biological Chemistry., 281(6), pp. 3290-3296.

MORGAN, M. J. and LIU, Z. G., 2011. Crosstalk of reactive oxygen species and NF-κB signaling. Cell research, 21(1), pp. 103-115.

NAKAMURA, T., NAKAMURA, H., HOSHINO, T., UEDA, S., WADA, H. and YODOI, J., 2005. Redox regulation of lung inflammation by thioredoxin. Antioxidants & redox signaling, 7(1-2), pp. 60-71.

NATHAN, C. and CUNNINGHAM-BUSSEL, A., 2013. Beyond oxidative stress: an immunologist's guide to reactive oxygen species. Nat Rev Immunol, 13(5), pp. 349-361.

ROBINSON, M. W., HUTCHINSON, A. T., DALTON, J. P. and DONNELLY, S., 2010. Peroxiredoxin: a central player in immune modulation. Parasite immunology, 32(5), pp. 305-313.

SCHREIBER, J., JENNER, R. G., MURRAY, H. L., GERBER, G. K., GIFFORD, D. K. and YOUNG, R. A., 2006. Coordinated binding of NF-κB family members in the response of human cells to lipopolysaccharide. Proceedings of the National Academy of Sciences, 103(15), pp. 5899-5904.

TORTI, F. M. and TORTI, S. V., 2002. Regulation of ferritin genes and protein. Blood, 99(10), pp. 3505-3516.

1. A method of treating inflammatory or cell proliferation disorders of the lungs in a patient which includes:
administering to the patient in need thereof a therapeutically effective amount of a synthetic pulmonary surfactant composition, wherein the synthetic pulmonary surfactant composition comprises:
a lipidaceous carrier; and
a peptide complex of poly-L-lysine or a pharmaceutically acceptable salt thereof and
poly-L-glutamic acid or poly-L-aspartic acid or a pharmaceutically acceptable salt thereof, the peptide complex having a charge-neutralized region and a positively-charged region.

2. The method as claimed in claim 1, wherein the salt of poly-L-lysine is poly-L-lysine.HBr of the formula (I) and n ranges from 100 to 135 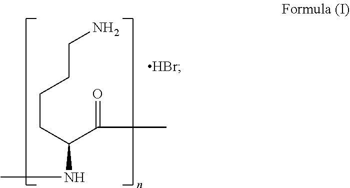
and wherein the salt of poly-L-glutamic acid is poly-L-glutamic acid sodium salt of the formula (II) and x is at least 50

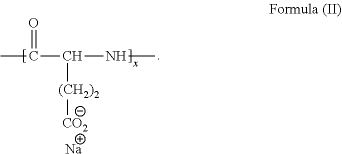

3. (canceled)

4. The method as claimed in claim 1, wherein the poly-L-lysine chain is longer than the poly-L-glutamic acid or poly-L-aspartic acid chain by at least 17 residues.

5. The method as claimed in claim 1, wherein the synthetic pulmonary surfactant composition comprises:
dipalmitoyl phosphatidylcholine (DPPC);
phosphatidylglycerol (PG);
hexadecanol;
tyloxapol;
poly-L-lysine.HBr;
poly-L-glutamic acid sodium salt;
sodium chloride; and
a pharmaceutically acceptable carrier.

6. The method as claimed in claim 1, wherein the treatment of inflammatory or cell proliferation disorders of the lungs occurs by the inhibition of the secretion of pro-inflammatory cytokines by alveolar macrophages in the presence of the synthetic pulmonary surfactant composition.

7. The method as claimed in claim 6, wherein the pro-inflammatory cytokines are TNF-α, IL-1β, IL-6 and KC/GRO.

8. The method as claimed in claim 1, wherein the cell proliferation disorder is lung cancer.

9. The method as claimed in claim 8, wherein the lung cancer is a lung adenocarcinoma.

10. The method as claimed in claim 8, wherein a chemotherapeutic agent is co-administered with the synthetic pulmonary surfactant composition.

11. (canceled)

12. (canceled)

13. (canceled)

14. (canceled)

15. (canceled)

16. (canceled)

17. (canceled)

18. The method as claimed in claim 1, wherein the therapeutically effective amount of the synthetic pulmonary surfactant composition is administered to the lungs via intubation, direct pulmonary administration or inhalation.

19. A method of treating a lung infection in a patient which includes:
administering to the patient in need thereof a therapeutically effective amount of a synthetic pulmonary surfactant composition and drug combination, wherein the synthetic pulmonary surfactant composition comprises:
a lipidaceous carrier;. and
a peptide complex of poly-L-lysine or a pharmaceutically acceptable salt thereof and poly-L-glutamic acid or poly-L-aspartic acid or a pharmaceutically acceptable salt thereof, the peptide complex having a charge-neutralized region and a positively-charged region.

20. The method as claimed in claim 19, wherein:
the salt of poly-L-lysine is poly-L-lysine.HBr of the formula (I) and n ranges from 100 to 135 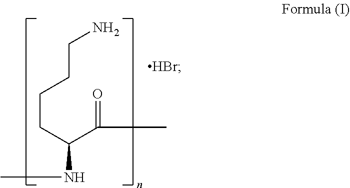
and
wherein the salt of poly-L-glutamic acid is poly-L-glutamic acid sodium salt of the formula (II) and x is at least 50

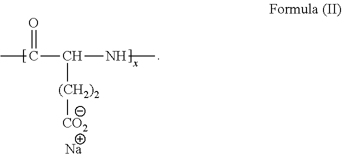

21. (canceled)

22. The method as claimed in claim 19, wherein the poly-L-lysine chain is longer than the poly-L-glutamic acid or poly-L-aspartic acid chain by at least 17 residues.

23. The method as claimed in claim 19, wherein the synthetic pulmonary surfactant composition comprises:
dipalmitoyl phosphatidylcholine (DPPC);
phosphatidylglycerol (PG);
hexadecanol;
tyloxapol;
poly-L-lysine.HBr;
poly-L-glutamic acid sodium salt;
sodium chloride; and
a pharmaceutically acceptable carrier.

24. The method as claimed in claim 19, wherein the synthetic pulmonary surfactant composition and drug provide dual immunomodulatory effects and the synthetic pulmonary surfactant composition acts as a permeabilizing agent of cell membranes that increases the permeability of the drug across the membrane.

25. The method as claimed in claim 19, wherein a therapeutically effective amount of the synthetic pulmonary surfactant composition and drug combination is administered to the lungs via intubation, direct pulmonary administration or inhalation.

26. (canceled)

27. The method as claimed in claim 19, wherein the lung infection is a bacterial or viral infection associated with one or more of tuberculosis (TB), pneumonia, cystic fibrosis and/or other diseases or diseased states.

28. The method as claimed in claim 19, wherein the drug is selected from one or more of tobramycin, Isoniazid (INH), Moxifloxacin, Ofloxacin, Pyrazinamide, Linezolid, amoxicillin, and ceftazidime.

29. The method as claimed in claim 19, wherein the drug is an antibiotic effective against Gram positive bacteria and suitable for use in the treatment of a Mycobacterium tuberculosis infection (TB).

30. (canceled)

31. (canceled)
